(19) United States
(12) Patent Application Publication
(10) Pub. No.: US 2020/0305970 A1
(43) Pub. Date: Oct. 1, 2020

(54) ELECTROPHYSIOLOGY PROCEDURE WITHOUT IONIZING RADIATION IMAGING

(71) Applicant: Navix International Limited, Road Town (VG)

(72) Inventors: Shlomo BEN-HAIM, Milan (IT); Yitzhack SCHWARTZ, Haifa (IL)

(73) Assignee: Navix International Limited, Road Town (VG)

(21) Appl. No.: 16/769,591

(22) PCT Filed: Dec. 5, 2018
(86) PCT No.: PCT/IB2018/059672

§ 371 Date: Jun. 4, 2020

Related U.S. Application Data
(60) Provisional application No. 62/594,637, filed on Dec. 5, 2017.
(60) Provisional application No. 62/667,653, filed on May 7, 2018.

(51) Int. Cl. A61B 18/14; A61B 5/00; A61B 5/02

(52) U.S. Cl. CPC A61B 18/1492; A61B 5/6852; A61B 2018/0022; A61B 5/6851; A61B 5/02007

(57) ABSTRACT

Methods and systems for position determination of an intrabody probe, targets of an intrabody probe, and or actions to be performed using an intrabody probe are described. In some embodiments, an anatomy being navigated and/or mapped is described by a rule-based schema relating different anatomically identified structures to one another according to their ability to help identify and/or locate one another. Additionally, in some embodiments, data recorded from the intrabody probe is processed according to schema rules in order to provide anatomical identification of the anatomical region which the intrabody probe is measuring, optionally without performing detailed mapping, and/or prior to the availability of detailed mapping of anatomical geometry.

### RELATED APPLICATIONS

This application claims the benefit of priority under 35 USC § 119(e) to U.S. Provisional Patent Application No. 62/667,653 entitled “VERSATILE IMAGING”, filed on May 7, 2018; and to U.S. Provisional Patent Application No. 62/594,637 entitled “EP PROCEDURE WITHOUT X-RAY”, filed on Dec. 5, 2017; the contents of which are incorporated herein by reference in their entirety.

This application is related to PCT Patent Application No. PCT/IB2017/057185, filed on Nov. 16, 2017, entitled “ESOPHAGUS POSITION DETECTION BY ELECTRICAL MAPPING”, and U.S. Provisional Patent Application No. 62/504,339, filed on May 10, 2017, entitled “PROPERTY-AND POSITION-BASED CATHETER PROBE TARGET IDENTIFICATION”. The contents of the above applications are all incorporated herein by reference in their entirety.

### FIELD AND BACKGROUND OF THE INVENTION

The present invention, in some embodiments thereof, relates to the field of navigation of body cavities by intra-body probes, and more particularly, to determination of intra-body probe position, for example during navigation of body cavities.

Several medical procedures in cardiology and other medical fields comprise the use of intrabody probes such as catheter probes to reach tissue targeted for diagnosis and/or treatment while minimizing procedure invasiveness. Early imaging-based techniques (such as fluoroscopy) for navigation of the catheter and monitoring of treatments continue to be refined, and are now joined by techniques such as electrical field-guided position sensing systems.

### SUMMARY OF THE INVENTION

There is provided, in accordance with some embodiments of the present invention, a method of guiding a catheterization procedure in a patient using electrical field sensing by an electrode probe, the method comprising: measuring effects of electrical field interaction with tissue of the patient using the electrode probe; generating guidance based on the measuring and indicative of information providing a reference for user actions moving the electrode probe from an insertion location into the patient to a target along a planned catheter route; and displaying the guidance during the measuring.

In some embodiments, the guidance is adapted according to a current position of the probe, using the measuring.

In some embodiments, the measuring is performed as the electrode probe moves from the insertion location into the patient to the target.

In some embodiments, the guidance is updated based on the measuring during the movement of the electrode probe from the insertion location into the patient to the target.

In some embodiments, the guidance is updated based on position data indicating positions of the electrode probe.

In some embodiments, the electrical fields alternate at one or more frequencies between 10 kHz and 10 MHz.

In some embodiments, the catheterization is for treating the target.

In some embodiments, the target is in a body cavity.

In some embodiments, the displaying comprises displaying guidance for application of treatment in the body cavity.

In some embodiments, the displaying guidance for application of the treatment is performed without the use of an imaging sensor external to the patient.

In some embodiments, the guidance indicates a shape of the body cavity determined using the measuring.

In some embodiments, the shape is determined without the use of an imaging sensor external to the patient.

In some embodiments, the target is a location of a body region tested along the planned catheterization route, and the measuring comprises measuring at the body region.

In some embodiments, the measuring at the body region comprises inspection of the body region for at least one of: plaque, growth presence, and vascular stenosis.

In some embodiments, the displaying guidance for movement comprises displaying one or more images of the surrounding of the probe.

In some embodiments, the one or more images are generated using the electrical field measurements made by the electrode probe.

In some embodiments, the method comprises analyzing gradients of the electrical fields measured by the electrode probe to generate the one or more images.

In some embodiments, the method is carried out without using X-ray radiation during the measuring.

In some embodiments, the determining the shape of the body cavity includes reconstruction of a 3-D shape of the body cavity.

In some embodiments, the method comprises reconstructing body tissue lumen shapes from position data indicating positions of the electrode probe during the displaying guidance for movement, wherein all position data used in the reconstructing are obtained from the measuring.

In some embodiments, the electrode probe is at a distal end of a catheter or guidewire.

In some embodiments, at least some of the electrical fields measured in the measuring are transmitted by electrodes of the electrode probe.

In some embodiments, the displaying guidance is performed without the use of an imaging sensor external to the patient.

In some embodiments, the displaying guidance for movement is performed without the use of contrast medium.

In some embodiments, the measuring is performed as the electrode probe moves from the insertion location into the patient to the target, and comprises measuring of a vascular obstruction, and the displayed guidance indicates the vascular obstruction.

In some embodiments, the vascular obstruction comprises at least one of the group consisting of: plaque, a vascular stenosis, and a growth.

In some embodiments, the measuring is performed as the electrode probe moves from the insertion location into the patient to the target, and comprises measuring of a vascular branch, and the displayed guidance indicates the vascular branch.

In some embodiments, the target is in a chamber of a heart.

In some embodiments, the measuring comprises measuring during each of: introduction of the probe to tubular lumen; navigation of the probe to a vascular branch or vascular obstruction; and navigation of the probe past the vascular branch or vascular obstruction to a body cavity comprising the target.

In some embodiments, no X-ray radiation is used from a time of insertion of the probe to the patient to the withdrawing.

There is provided, in accordance with some embodiments of the present invention, a method of guiding a catheterization procedure in a patient using electrical field sensing by an electrode probe, the method comprising: measuring effects of electrical field interaction with tissue of the patient using the electrode probe; generating guidance based on the measuring and indicative of information providing a reference for user actions moving the electrode probe from a vascular obstruction or branch encountered by the electrode probe after an insertion of the probe into the patient to a target along a planned catheter route; and displaying the guidance during the measuring.

In some embodiments, the measuring is performed as the electrode probe moves from the insertion location into the patient to the target.

In some embodiments, the guidance is updated based on the measuring during the movement of the electrode probe from the vascular obstruction or branch to the target.

In some embodiments, the guidance is updated based on position data indicating positions of the electrode probe.

In some embodiments, the vascular obstruction comprises at least one of the group consisting of: plaque, a vascular stenosis, and a growth.

In some embodiments, the vascular obstruction or branch is encountered after the insertion and before any other vascular obstruction or branch encountered during the catheterization procedure.

In some embodiments, the vascular obstruction or branch is in a femoral blood vessel.

In some embodiments, the target is in a chamber of a heart.

In some embodiments, the catheterization is for treating the target.

In some embodiments, the target is a location of a body region tested along the planned catheterization route, and the measuring comprises measuring at the body region.

In some embodiments, the displaying guidance for movement comprises displaying one or more images of the surrounding of the probe.

In some embodiments, the method carried out without using X-ray radiation during the measuring.

In some embodiments, the electrode probe is at a distal end of a catheter or guidewire.

In some embodiments, at least some of the electrical fields measured in the measuring are transmitted by electrodes of the electrode probe.

In some embodiments, the displaying guidance is performed without the use of an imaging sensor external to the patient.

In some embodiments, the displaying guidance for movement is performed without the use of contrast medium.

In some embodiments, the measuring is performed as the electrode probe moves from the insertion location into the patient to the target, and comprises measuring of a vascular obstruction, and the displayed guidance indicates the vascular obstruction.

There is provided, in accordance with some embodiments of the present invention, a method of allocating operation rooms to operation procedures, the method comprising: selecting an X-ray unshielded room; and allocating the selected room to a catheterization procedure, thereby freeing X-ray shielded rooms to operation procedures for which X-ray shielding is essential.

In some embodiments, the method comprises selecting an X-ray unshielded room that does not contain an X-ray machine.

There is provided, in accordance with some embodiments of the present invention, a system for guiding a catheterization process, the system comprising: a radiation source, configured to generate non-ionizing electromagnetic radiation; a catheter comprising an electrode probe configured to apply non-ionizing electromagnetic radiation generated by the radiation source to a penetrated blood vessel of a patient; a data analyzer, configured to generate guidance for movement of the catheter from a vascular obstruction or branch encountered by the electrode probe after an insertion of the probe into the patient to a target beyond the vascular obstruction or branch and along a planned catheter route, based on measurements indicative of interactions of tissue near the electrode probe with the non-ionizing electromagnetic radiation applied by the electrode probe; and a catheterization system configured to guide the electrode probe inside the patient, the catheterization system being arranged to be operated by a user when the user is receiving the guidance generated by the data analyzer.

In some embodiments, the data analyzer is configured to generate the guidance for movement independently of measurements taken during the catheterization other than the measurements indicative of interactions of tissue near the electrode probe with the non-ionizing electromagnetic radiation generated by the radiation source.

In some embodiments, the electrode probe is at a distal end of a catheter.

In some embodiments, the system does not require X-ray shielding in order to operate under applicable safety regulations.

In some embodiments, the non-ionizing radiation is of electromagnetic fields alternating at one or more frequencies between 10 kHz and 10 MHz.

There is provided, in accordance with some embodiments of the present invention. a catheterization room comprising: a processor connected to: a display, an input for receiving from an intra-body electrode probe measurements of electrical fields, and a data analyzer connected to the input and configured to generate an image from the readings of the electrical fields; a support for a patient to be operated in the operation room; a catheterization system configured to guide a catheter inside a patient supported by the support, arranged to be operable by a physician when the physician is viewing the display; and walls defining the catheterization room, the walls being X-ray penetrable.

In some embodiments, the catheterization room comprises at least one X-ray penetrable window.

In some embodiments, the electrical fields are non-ionizing.

In some embodiments, the electrical fields are alternating fields, alternating at one or more frequencies of between 10 kHz and 10 MHz.

There is provided, in accordance with some embodiments of the present disclosure, a method of performing a treatment procedure in a patient without the use of X-ray, contrast medium injection, or ultrasound (‘US’) imaging. Optionally, the treatment procedure is an electrophysiology (EP) procedure in a patient heart; for example: pulmonary vein isolation (PVI) ablation. In some embodiments, the entire procedure (including for example: inserting a catheter to the patient heart, mapping, navigating and ablating) is performed without the use of X-ray, contrast medium injection, or ultrasound imaging.

There is provided, in accordance with some embodiments of the present disclosure, a method of monitoring the position of an electrode probe using electrical field sensing during performance of a treatment procedure in a patient using the electrode probe, the method comprising: displaying guidance for movement of the electrode probe from an insertion location into the patient to a body cavity of the patient comprising tissue targeted for treatment; determining the shape of the body cavity; displaying guidance for application of the treatment in the body cavity; and measuring electrical fields within the patient using the electrode probe during the displaying guidance for movement, determining, and displaying guidance for application of the treatment; wherein the displaying guidance for movement, determining, and displaying guidance for application of the treatment are all performed based on the measuring.

In some embodiments, sensing for the imaging is performed using electrodes of the electrode probe.

In some embodiments, the determining the shape of the body cavity is performed using electrodes of the electrode probe.

In some embodiments, all sensing for the imaging is performed using electrodes of the electrode probe.

In some embodiments, the determining the shape of the body cavity includes reconstruction of a 3-D shape of the body cavity.

In some embodiments, all position data used in the reconstructing are obtained from the measuring.

In some embodiments, the electrode probe is advanced at the distal end of a catheter.

In some embodiments, at least some of the electrical fields used in the measuring are also transmitted by electrodes of the electrode probe.

In some embodiments, the displaying guidance for movement, determining, and displaying guidance for application of the treatment are all performed without the use of an imaging device external to the patient.

In some embodiments, the displaying guidance for movement, determining, and displaying guidance for application of the treatment are all performed without the use of X-ray imaging.

There is provided, in accordance with some embodiments of the present disclosure, a method of determining an anatomical identity of a first intrabody region using an intrabody probe, the method comprising: receiving an indication of a current operational context of the intrabody probe; receiving input data from the intrabody probe indicating one or more measured properties of the first intrabody region; selecting at least one rule for anatomical identification from an anatomical schema, wherein the at least one rule is selected based on the current operational context; and applying the at least one rule to the input data, to anatomically identify the first intrabody region.

In some embodiments, the method comprises: selecting a second at least one rule for anatomical identification from the anatomical schema, based on the current operational context; and applying the second at least one rule to identify a second intrabody region, based on a relationship between the second intrabody region and the first intrabody region, and the anatomical identification of the first intrabody region.

In some embodiments, the method comprises associating the anatomical identification to a map comprising a geometrical representation of the first intrabody region.

In some embodiments, the method comprises displaying the anatomical identification together with a display of the geometrical representation of the first intrabody region.

In some embodiments, the method comprises guiding navigation of the intrabody probe to the anatomically identified first intrabody region, based on the anatomical identification.

In some embodiments, the method comprises using the intrabody probe to perform an action upon the anatomically identified first intrabody region, based on the anatomical identification.

In some embodiments, the input data comprises only non-image data.

In some embodiments, the indication of a current operational context comprises is a non-image indication.

In some embodiments, the input data comprises electrical measurements of the intrabody region.

In some embodiments, the electrical measurements comprise voltage measurements.

In some embodiments, the electrical measurements comprise impedance measurements.

There is provided, in accordance with some embodiments of the present disclosure, a method of generating an estimator of an anatomical identity of an intrabody region, comprising: collecting input data from an intrabody probe; obtaining a plurality of indications from at least one skilled operator of the intrabody probe, wherein the indication are of an anatomical identity of intrabody regions present at different intrabody positions of the intrabody probe while the input data was collected; and processing the collected input data together with the plurality of indications to generate an estimator which anatomically identifies an intrabody region, based on new input data collected from an intrabody probe.

In some embodiments, the input data comprises electrical measurements of the intrabody region.

In some embodiments, the method comprises processing to generate a plurality of the estimators for assigning a corresponding plurality of different anatomical identifications, based on the input data and the plurality of indications.

In some embodiments, the processing comprises application of a machine learning method.

In some embodiments, the collecting comprises collecting estimator results from a second estimator produced according to the method above; wherein processing to generate the estimator comprises using the results collected from the second estimator.

There is provided, in accordance with some embodiments of the present disclosure, a method of crossing an interatrial septum, comprising: positioning an intrabody probe at multiple locations adjoining the interatrial septum while using the intrabody probe to measure an indication of a tissue property of the interatrial septum at each of the multiple locations; locating a crossing location on the interatrial septum, based on a difference at the crossing location in the measurements of the tissue property, compared to other locations; and moving the intrabody probe across the crossing location, based on the locating.

In some embodiments, the measured indication comprises an electrical field parameter affected by the indicated tissue property.

In some embodiments, the intrabody probe comprises a needle, and the indication of the tissue property is electrically sensed using the needle.

In some embodiments, the method comprises sensing a change in an electrical signal as the needle extends from a sheath to cross the crossing location, and displaying tenting movement of a simulated display of the interatrial septum in correspondence with the sensed change in the electrical signal.

In some embodiments, the moving the intrabody probe across the crossing location comprises ablating at the crossing location using the probe to weaken tissue at the crossing location.

In some embodiments, the method comprises using the same intrabody probe to perform another ablation in a heart chamber entered after crossing the crossing location.

There is provided, in accordance with some embodiments of the present disclosure, a method of verifying the placement of a cryoballoon, comprising: inserting at least one sensing electrode of an intrabody probe into an opening of a pulmonary vein; monitoring output from the sensing electrode; positioning a cryoballoon located on the same intrabody probe so that it occludes the opening; and detecting a rapid change in the output of the sensing electrode, corresponding to the time of occlusion of the opening.

In some embodiments, the occlusion of the opening is sufficient to block blood flow through the opening.

In some embodiments, the method comprises proceeding with an ablation procedure, based on confirmation from the detected rapid change that the cryoballoon is inserted to the vein opening entered by the sensing electrode.

In some embodiments, the method comprises proceeding with an ablation procedure, based on confirmation from the detected rapid change that the cryoballoon is in contact with tissue near the vein opening around an uninterrupted perimeter.

There is provided, in accordance with some embodiments of the present disclosure, an apparatus for determining an anatomical identity of a first intrabody region, the apparatus comprising: an interface configured to receive from a user of the apparatus an indication of a current operational context of an intrabody probe; an intrabody probe input for receiving input data from the intrabody probe indicating one or more measured properties of the first intrabody region; a memory storing a plurality of rules for determining the identity of the first intrabody region, each rule being associated with a respective operational context; and a processor configured to: select at least one rule from the memory based on operation context received through the interface; and apply the at least one rule to the input data, to determine the anatomical identity of the first intrabody region.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example, and for purposes of illustrative discussion of embodiments of the invention. In this regard, the description taken with the drawings makes apparent to those skilled in the art how embodiments of the invention may be practiced.

In the drawings:

FIG. 1A schematically represents a method of automatic anatomical identification of an intrabody target, and optionally automatic suggestion of a selected action on that target, according to some embodiments of the present disclosure;

FIG. 1B is a schematic flowchart of the use the method of FIG. 1A within the context of a procedure, according to some embodiments of the present disclosure.

FIG. 2A schematically illustrates a system for use in performing the methods of FIGS. 1A-1B, including a schematic representation of a patient body, according to some embodiments of the present disclosure;

FIG. 2B schematically represents inputs and operations of an estimator services module, according to some embodiments of the present disclosure;

FIG. 3A schematically represents selected anatomical relationships encoded by an anatomical schema, according to some embodiments of the present disclosure;

FIG. 3B illustrates some of the left atrium features mentioned in FIG. 3A in an “unwrapped” view of the left atrium, according to some embodiments of the present disclosure;

FIG. 3C is a schematic flowchart of the use of machine learning to establish at least some aspects of an anatomical schema, according to some embodiments of the present disclosure;

FIGS. 4A-4C schematically represent crossing by a catheter probe from a right atrium across an interatrial septum to a left atrium via a fossa ovalis, according to some embodiments of the present disclosure;

FIG. 5 is a schematic flowchart describing a method of locating a fossa ovalis, according to some embodiments of the present disclosure;

FIG. 6 is a schematic flowchart describing a method of crossing a fossa ovalis using an electrically monitored needle, according to some embodiments of the present disclosure;

FIGS. 7A-7B schematically represent stages in cryoablation including insertion of a lasso catheter probe into a pulmonary vein of a left atrium, and conversion of blood flow into blocked flow as a cryoballoon is pressed firmly up against the ostium leading into pulmonary vein, according to some embodiments of the present disclosure;

FIG. 8 is a schematic flowchart describing a method for electrical monitoring of the flow blockage shown in FIGS. 7A-7B, according to some embodiments of the present disclosure;

FIGS. 9A-9D schematically represent test results of the method of FIG. 8, according to some embodiments of the present disclosure;

FIGS. 10A-10B respectively represent visual results of cryoablation in vitro on a muscle tissue preparation (FIG. 10A), and dielectric assessment of the same results (FIG. 10B) which reveals a potential gap in the apparently well-ablated region, according to some embodiments of the present disclosure;

FIG. 11 is a schematic flowchart describing a method for single-electrode transseptal penetration from the right to the left atria, followed by ablation within the left atrium, according to some embodiments of the present disclosure;

FIG. 12 is a flowchart describing a method of using an electrode probe to navigate to and treat a target in a body tissue cavity, according to some embodiments of the present disclosure;

FIG. 13 schematically illustrates components and body structure elements described in relation to the method of FIG. 12, according to some embodiments of the present disclosure;

FIGS. 14A-14C illustrate a result of electrical mapping of a phantom left atrium (a plastic resin model immersed in a water-filled tank), with and without a phantom aorta (saline-filled syringe) located alongside, according to some embodiments of the present disclosure;

FIGS. 15A-15C schematically illustrate stages in the insertion to a body of an electrode-equipped guide-wire configured for electrical imaging, according to some embodiments of the present disclosure;

FIG. 16 schematically illustrates an electrode-equipped guidewire configured for electrical imaging, shown in relation to an at least partially obstructed blood vessel, according to some embodiments of the present invention.

FIG. 17 schematically illustrates a guidewire equipped with electrodes, according to some embodiments of the present disclosure;

FIG. 18 is a flowchart describing use of a guidewire for imaging, according to some embodiments of the present disclosure; and

FIG. 19 is a flowchart describing use of various electrode-based imaging tools during the course of a medical procedure, according to some embodiments of the present invention.

### DESCRIPTION OF SPECIFIC EMBODIMENTS OF THE INVENTION

The present invention, in some embodiments thereof, relates to the field of navigation of body cavities by intra-body probes, and more particularly, to determination of intra-body probe position, for example during navigation of body cavities.

### Overview

An aspect of some embodiments of the present invention relates to the performance of an entire minimally invasive procedure using intrabody probes, and relying on electrical field sensing to provide information about the immediate environment (e.g., lumenal environment) of the intrabody probes, preferably while eliminating reliance on imaging based on sensing other than electrical field sensing. In some embodiments, the intrabody probes are introduced intrabody during a catheter procedure (equivalently, a “catheterization” procedure).

As used herein, the term “imaging” refers to the production of a 2-D and/or 3-D representation (an image) of the spatial structure of a target (the target comprising, in some embodiments, anatomical structure of a body), through use of a sensor (also referred to herein as an “imaging sensor”) that measures image data correlating to the represented spatial structure of the target. In some embodiments, the measured image data are structured into the representation of spatial structure using knowledge of the conditions of the data measurement (e.g., structuring comprises the performing of calculated transformations on the data), and/or directly structured into the representation because of the way that the image data are measured (e.g., as in the case of photons focused on a photosensitive film using lenses). Relevant conditions of the image data measuring may include, for example: how the sensor is shaped and/or moves; sensitivity characteristics of the sensor; how radiant energy that interacts with the target (if used) is originated, directed, and/or behaves in response to target interactions; and/or how the sensor itself behaves in response to its interactions with the target (for sensing that includes direct probe/target interactions such as physical contact).

More particularly, “remote imaging” methods, herein, comprise imaging methods in which the sensor measures by detecting radiant energy while located at positions away from the positions at which features of the imaged target structure and the radiant energy interact. A characteristic of remote imaging methods is that movement (if any) of the sensor is not dictated by the detailed shape of the imaged target structure. “Contact imaging” methods, herein, comprise imaging methods in which the sensor measures while located in contact with and/or moved in ways constrained by the contours of imaged target structure, the features of which imaged structure are being measured as the features existing at the locations of the sensor.

Furthermore, “external” imaging methods, herein, involve data measured from a sensor (an external imaging sensor) which is outside the body being imaged, and “internal” imaging methods involve a sensor which is inside the body being imaged. An internal imaging method may more particularly be performed internal to a body lumen being imaged. An imaging method can be both internal and remote; for example, a sensor can be placed inside a lumen but out of contact with a wall of the lumen, and perform sensing for remote imaging of the lumenal wall.

Herein, the term “probe” (in the context of imaging) means a device which is configured with at least one sensor, and is operated for sensing during imaging while in contact with and/or internal to a body. An “electrode probe” is a probe that carries at least one electrode used for electrical field sensing.

As used herein, the phrase “electrical field sensing” means measuring, using an electrode, voltage potential and/or electrical current induced by some electrical field, and as it is measured with the electrode at a certain position. In some embodiments, the electrical field is alternating, for example, at frequencies of between about 10 kHz and about 10 MHz, for example, about 18.5 kHz.

The voltage potential and/or electrical current, or a ratio between them (e.g., an impedance) measured with the electrode at a certain position may be referred to herein as the state of the electrical field “at” the position of the electrode. It is to be understood that electrical field sensing is generally with respect to some reference, such as another electrode at a reference voltage potential. The reference may also comprise a physical system. For example, impedance is measured, in some embodiments, relative to an electrical environment including: body tissue; a probe and a particular position of the probe in that body tissue; and other electrical circuit components such as electrodes, wires, and signal generators and/or amplifiers. Measured impedances (for example) may be said herein to be impedances of and/or at particular locations. This linguistic convention may be understood as indicating that different position-characteristic measurements of impedance are made with a probe electrode at different positions, using some otherwise substantially fixed, well-controlled and/or well-characterized: electrical circuit, operating settings of that electrical circuit, and electrical environment. The electrical environment includes, for example, tissue with its characteristic dielectric properties, and the quality of electrical contact of electrodes in contact with the tissue. The same linguistic convention attributing measurements to positions also applies to other measurements, for example, measurements described as measurements of voltage.

In some embodiments, electrical field sensing is used in imaging. In some embodiments, the imaging combines separate position data (giving the position of the electrode) and measurement data (giving a measurement of at least one electrical field at the position). In some embodiments, measurement data also act as position data, e.g., insofar as measurement data values from electrical field sensing change as a predictable and/or discoverable function of measurement position. In some embodiments, position is the primary parameter being sensed, and an image is created using measurement positions as an indication of structure—for example, the shape of a lumen is imaged by sensing many different positions of an electrode moving within the lumen, e.g., exploring along physical boundaries of the lumen.

Herein, certain imaging methods are said to include and/or be used in the production of a “reconstruction” of the spatial structure of a volume, for example, of the target. Herein, a reconstruction comprises a representation of the spatial structure that relates different measurements to a common underlying shape. In comparison to an image: an image, as such, represents “where structure occurs”, e.g., spatially arranged pixels of varying intensity. A reconstruction is a data structure, for example for example an image or model, that relates structure measured at different positions to a shape that spans those positions. Typical results of reconstruction are representations of boundaries, surfaces, and/or volumes (e.g., of lumens and/or lumenal surfaces of internal body organs).

Although reconstruction may be performed after the imaging, in some instances, a process of imaging itself is closely tied to a method of reconstruction. For example, the transformations used to create an image may rely on assumptions about the existence of continuous shapes in the body such as boundaries, surfaces and/or volumes; for example, to constrain the spatial positions assigned to ambiguous data to physically plausible alternatives. Transformation use can also assist, for example, removing noise, and/or rejecting outliers when producing an image.

A reconstruction, in some embodiments, may be a direct result of a process of image segmentation—for example, segmentation comprising a threshold operation that binarizes image measurements as representing an “inside” or “outside” of some structure. There is often additional or alternative processing performed during a process of segmentation to increase accuracy and/or overcome ambiguity, for example, use of methods for edge detecting, region growing, data clustering. In another example, an image created from a point cloud may be used to reconstruct a volume, for example, by a rolling-ball algorithm that seeks to find an envelope with contours that encapsulate the point cloud in a minimal volume while also remaining smooth. Optionally an earlier-stage reconstruction such as a segmented image is processed to create a further reconstruction, e.g., a mesh or other parameterized 3-D shape is determined which fits a segmented shape.

Herein the term “guidance” is used in the context of the movement of a probe such as a part of a guidewire or a catheter. “Guidance” in such contexts refers to information provided to a user, which a user uses as a reference to perform actions which bring the probe to a target (which may be a predetermined location, e.g., of a body cavity) along a route. Guidance based on measuring performed by the probe is adapted according to the current position of the probe to serve as a reference, using the measuring. The guidance, in some embodiments, is machine generated and/or machine displayed. In some embodiments, the route of the probe is planned from an insertion point into a body to a target within the body. For example, the route is planned from an insertion site at a femoral vein or artery through characteristic patterns of branching blood vessels, and up to a specific chamber of the heart. Guidance, in some embodiments, may be displayed when the probe is at any suitable point along the route, and optionally all along the route. The suitable points may be points in space and/or in time. In some embodiments, the suitable points may be pre-defined, for example, as anatomical landmarks, and/or as points where a predetermined change in the received electrical fields occurs. Alternatively or additionally, the suitable points may be predefined by time periods, for example, a predetermined time period after entering the femoral artery, a predetermined time after encountering a landmark, etc. The landmarks may include vascular branches, organs in the vicinity of the catheter probe, and the like. Herein, examples are described primarily in relation to blood vessels. However, it should be understood that embodiments of the present disclosure also include catheter procedures along lymph vessels, fallopian tubes, urethra, ureter, and optionally direct insertion into a body cavity, e.g, via a needle in the back and/or an incision leading to an intraperitoneal space.

The target, in some embodiments, is an anatomical target. More particularly, the anatomical target is optionally a particular portion of a body lumen; for example, a heart chamber, a position within a heart chamber such as a contact with a particular heart wall portion, and/or vascular branch. In some embodiments, the guidance comprises an image indicating a shape of the target, and/or a shape of an anatomical region which the probe traverses on the way to the target. In some embodiments, the guidance comprises an indication of probe position in relation to indicated shapes. In some embodiments, the guidance comprises text, direction signals, sounds, haptic indications, iconic indications, and/or other presentations of information which the user senses.

Presentation of guidance is referred to herein as “displaying”, in the sense of “making manifest to the senses”, whether or not the displaying is visual in character. Examples of displaying guidance include:
Images, e.g., of vascular extents and/or cardiac chambers (optionally displayed together with a representation of a catheter probe);
Spoken or visually presented words, and/or direction arrows suggesting and/or indicating, e.g., direction of rotation, advance, retraction, and/or steering of a catheter or guidewire; and/or
Sounds, graphical icons and/or haptic (vibratory, for example) outputs indicating, e.g., probe contacts with lumenal walls, arrival at a target and/or waypoint, and/or steering to a favorable orientation.

Herein, guidance may be referred to as displayed “based on” measurements. Displaying an image and/or a reconstruction made using those measurements is an example of displaying guidance based on those measurements. In some embodiments, instructions (e.g., arrows) and/or indications derive from the measurements (optionally through an intermediate stage of image and/or reconstruction) by machine analysis of the measurements. For example, a certain type of impedance measurement change (during measuring) is optionally converted to an indication of a wall contact (displaying guidance).

A broad aspect of some embodiments of the present invention relates to use of catheter probe measurements to establish anatomical identity of intrabody regions, particularly intrabody regions in the vicinity of the probe.

Methods for determining the anatomical geometry of intrabody regions navigated by catheters have been described based on many different techniques; for example, CT imaging, X-ray angiographic imaging, MRI imaging, ultrasound imaging, electrical field-guided probe navigation, and magnetic field-guided probe navigation.

Some such methods build up an image of anatomical geometry based at least in part on data acquired on the fly during a catheterization procedure. For example, methods using electrical field-guided probe navigation may use electrically recorded data to build up an anatomical model which gradually increases in coverage, resolution, and/or accuracy as a procedure progresses. Accordingly, an operator may be presented with a need to perform procedure operations based on incomplete geometrical information. Moreover, and potentially even in situations where anatomical geometry is well-represented, an operator (particularly an inexperienced operator) may occasionally become confused in making an anatomical identification based on anatomical geometry information alone.

Misidentification of anatomical position, even if rare, potentially leads to serious complications. For example, trans-septal passage of an intracardiac catheter is a complicated intervention, which even after 200 cases of training has been associated with a risk of serious adverse events in the range of about 2%. One type of adverse event comprises penetration of the wrong part of the heart wall. Potentially, improvements in making the link between anatomical geometry and identification of that geometry as being of a particular (e.g., named) anatomical structure would help reduce such rates of complication.

Herein, a distinction is drawn between anatomical geometry and anatomical identity. Anatomical geometry comprises shapes of anatomy, and relationships among those shapes in the definition of larger structures. As examples of anatomical geometry: a heart chamber has a (dynamically changing) roughly globular shape, from which one or more tubular blood vessels extend; the heart chamber also is in fluid communication with another roughly globular-shaped heart chamber. Anatomical identity comprises assigning to an anatomical position (e.g., a position defined within a modeled anatomical geometry, for example, the position of a shape or any portion thereof, including a point-like position) an identity as belonging to a particular anatomically defined structure, such as a right atrium, pulmonary vein, or even more particularly, for example, as an interatrial septum, foramen ovale ostium of a pulmonary vein, atrial appendage, and/or another anatomical structure. Anatomical identity of a position can generally be deduced from a sufficiently complete representation of anatomical geometry (e.g., by consideration of shapes at the position itself and/or the relationship of the position to shapes in other, e.g., adjacent, positions). But the two are distinct; for example, it can be understood that a blood vessel may be accidentally misidentified even by an operator viewing a detailed model. Working from a partial model of anatomical geometry, anatomical identity may be still more ambiguous. Other information, for example as described herein, may augment and/or replace the use of anatomical geometry in establishing anatomical identity.

Unless otherwise indicated, anatomical identity is generally understood to refer to macroscopic anatomical structures (e.g., of a region being navigated by a catheter probe). These macroscopic structures optionally correspond to named anatomical parts. However, in some embodiments, anatomical identity is optionally made at least in part according to distinctions other than those of the standard anatomical nomenclature, which can be made from available data. For example, different anatomical identities may be assigned to regions with different tissue wall thicknesses, tissue texture, or other structural and/or positional differences which can be detected (e.g., by the use of dielectric measurements), but do not necessarily correlate with distinctions made by standard anatomical nomenclature.

An aspect of some embodiments of the present invention relates to automatic anatomical identification of an intrabody region based on combined inputs from a plurality of measurement sources.

In some embodiments, the plurality of measurement sources comprises at least one source giving positional information, and at least one source giving measurements of one or more tissue properties which vary at different positions (e.g., measured electrical impedance of a circuit with an electrode at a position, and/or S matrix of an electrode array at the position).

An aspect of some embodiments of the present invention relates to the use of supervised machine learning to create one or more data structures useful in automatic anatomical identification of an intrabody region, and/or the provision of automatic indications of procedure actions to be performed in those regions.

In some embodiments, the one or more data structures include information describing alternate anatomical configurations which may be encountered during a procedure. Optionally, identification of one or more particular alternate anatomical configurations is further linked to automatic indication (e.g., recommendation) of procedure changes to potentially adapt procedure actions to the specific exigencies of an alternate anatomical configuration. In some embodiments, the automatic indications are produced based on supervised machine learning.

Some practitioners especially skilled in a procedure can identify targets, appropriate times, and/or alternatives for procedure actions with a high probability of success compared to peers. It would be of potential benefit to embed aspects of this skill in an automatic advisory system for use by less-skilled practitioners. In some procedures, for example, intervention procedures performed over catheter by indirect visualization, nearly all of the inputs (and many of the outputs) generated during a procedure are available in the same digital form originally available to practitioner. This condition provides an opportunity for expert skill capture to an automatic system, based on supervised learning.

In some embodiments, the digital records of a plurality of catheter procedures are used, together with supervised machine learning, to produce an automatic advisory system linking different situational specifics to different suggested actions. For example, all data presented to a skilled practitioner before some procedure action (and/or during the procedure action) are treated as inputs, while subsequent commanded movements and other actions are treated as outputs which suggest what is to be done, when to do it, and/or to what degree to do it.

Optionally, in some embodiments, a skilled practitioner provides additional indications (narration, for example), describing features of their judgments and/or intentions which may not be inherently visible in their recorded actions. Optionally, procedure records (with or without supplementary annotations from a practitioner) are subjected to further markup before use in machine learning, for example to divide and/or label epochs within the procedure record, and/or to change the weighting of different aspects of recorded information (e.g., if the skilled practitioner has highlighted some feature during the procedure as important to decision making, and/or if there is some aspect of procedure action timing, extent and/or degree which should be a subject of particular focus for the machine learning). Optionally, post-procedure data (for example, procedure outcome results) are also provided as part of the machine learning input.

In some embodiments, machine learning is used to advise a procedure practitioner on the locations of heart structures. For example, in intervention to correct a defective heart valve, the atrial ventricular ring to which the mitral and the tricuspid valves are attached is a significant target. In some embodiments, a locatable intrabody probe (for example, a catheter probe) has at least one electrode. An AC current is injected from each electrode, optionally at a plurality of frequencies, or otherwise distinguished, to allow separate identification of the electrodes used. The corresponding voltages generated on the same and/or other electrodes are recorded and processed by a Processing Unit (PU). These data comprise an example of conditions used within a learning data set. Optionally, an expert practitioner identifies signal recorded at certain positions as corresponding to a certain type of target (or non-target; that is, a region excluded from being the subject of a certain procedure action). This identification can be, for example, by actual actions performed (e.g., in a transseptal penetration, the fossa ovalis is selected for penetration—so the readings for the area actually penetrated implicitly are identified as pertaining to a fossa ovalis). Additionally or alternatively, the expert practitioner explicitly tags regions based on their own judgments.

In some embodiments, machine learning for this example uses input data in the matrices of the S₁₁, S₁₂ . . . Sᵢⱼ of the electrodes in different frequencies as well as the location of the probe relative to a known fiducial. An element Sᵢⱼ of an S matrix is a number, optionally a complex number, describing a ratio between an electrical field of a given frequency going through antenna i into the surroundings and an electrical field of the same frequency going at the same time through antenna j from the surroundings, when each antenna transmits an electrical field of a distinct frequency, e.g., in the radio frequency range of the electromagnetic spectrum. Optionally, the input data is provided for machine learning after normalization to correct for inter-patient variability. Expert actions and/or expert-provided observations provide the supervisory training feedback that relates the input data to particular cases, and serves as a basis for machine learning of association between input data and corresponding expert evaluations. After the machine learning result is validated as producing correct evaluations and/or action recommendations in response to data on parts of which the machine was trained, the learning result may be used to evaluate and/or recommend actions in response to new input.

An aspect of some embodiments of the present invention relates to providing of procedure guidance based on automatic anatomical identifications within an intrabody region.

In some embodiments, a procedure being guided comprises cryoablation. In some embodiments, a cryoballoon is used to ablate a closed line of tissue, for example, surrounding an entrance of a pulmonary vein to the left atrium. In some such embodiments, it is a potential advantage to have an indication of when the cryoballoon closes off flow through the pulmonary vein, since such blockage of flow potentially indicates that fully circumferential contact has been made by the balloon, so that a gap-free ablation line can be formed.

In some embodiments, procedure guidance includes detection (and indication to a user) of changes in sensed voltage by one or more electrodes located within a pulmonary vein as a cryoballoon configured for use in cryoablation closes off flow through the pulmonary vein.

Optionally, automatic procedure guidance is developed using techniques of machine learning. In some embodiments, experts indicate during a procedure, or during analysis of a replay of a procedure, when flow is blocked; and the machine learns relations between such indications and electrical potential readings. Results of the training may then be used to procedure guidance by following in real time changes in electrical potential detected by electrodes during a similar procedure carried out by a novice, and indicating when full blockage is achieved. In some embodiments, the system may be trained to identify actions to be taken once the flow blockage is achieved, and recommend these actions to the novice.

In some embodiments, a procedure being guided comprises penetrating the interatrial wall by an ablation catheter. In some embodiments, an electrode probe is passed over the interatrial wall while making dielectric measurements. Thinner walls are observed to have different dielectric properties than thicker walls. Optionally, position of thinning (or actual holes) near the center of the interatrial wall are treated as representing a target region across which an ablation probe is to penetrate the interatrial wall.

In some embodiments, a procedure to be guided comprises determining a location of a valve plane (e.g., in preparation for valvular treatment), and/or determining a location of an opening into the coronary sinus (e.g., in preparation for cannulation of the coronary sinus).

Optionally, automatic procedure guidance is developed using techniques of machine learning. In a learning stage, in some embodiments, an expert marks when a catheter is at a target position (e.g., the valve plane or the opening in the coronary sinus). The machine is trained to distinguish between readings of the electrodes at the target position and readings of the electrodes off the target positions. Then, in another procedure, the results of the training may be used to identify when the catheter is at the target position based on readings received from electrodes on the catheter.

For purposes of description, principles of the invention are described herein with respect to detailed embodiments relating to mapping and/or navigation of an intrabody probe (e.g., a catheter probe) within a cardiovascular system. In some embodiments, the mapping and/or navigation is performed in the context of a cardiac intervention, for example: cardiac electrophysiological treatment, cardiac vascular treatment, and/or cardiac structural heart disease treatment (for example valvular treatments). It should be understood that in some embodiments, principles of the invention are applied, changed as necessary as may be understood based on the provided examples, to another medical intervention; for example: surgery, colonoscopy, biopsy, oncology surgery, orthopedic disk surgery, and/or plastic surgery.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

Method of Targeting and/or Action Selection by Anatomical Identification

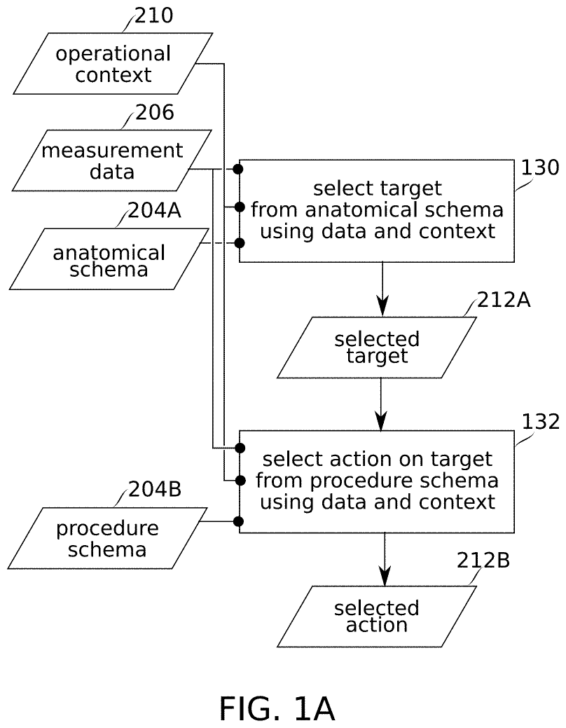

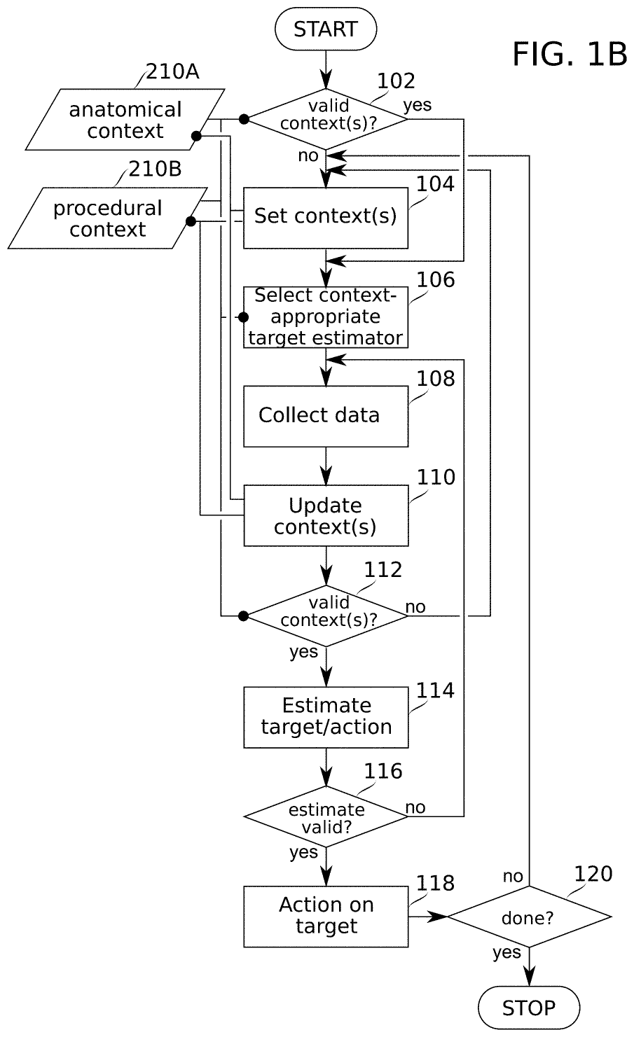

Reference is now made to FIG. 1A, which schematically represents a method of automatic anatomical identification of an intrabody target, and optionally automatic suggestion of a selected action on that target, according to some embodiments of the present disclosure.

At block 130, in some embodiments, the flowchart begins with selection of a target of a catheter operation. Inputs to block 130, in some embodiments, include operational context 210, measurement data 206, and anatomical schema 204A. These inputs to block 130 are also discussed in relation to other figures herein; in particular FIGS. 1B, 2A-2B and 3A-3B. The output of block 130, in some embodiments, is a selected target 212A; wherein the selected target 212A is selected from targets defined in the anatomical schema 204A using the measurement data 206 and the operational context 210. The implementation of the selecting is largely governed by features of the data structure comprising anatomical schema 204A, which describe how operational context 210 and measurement data 206 are to be used, as now described in the following brief overviews.

Operational Contexts

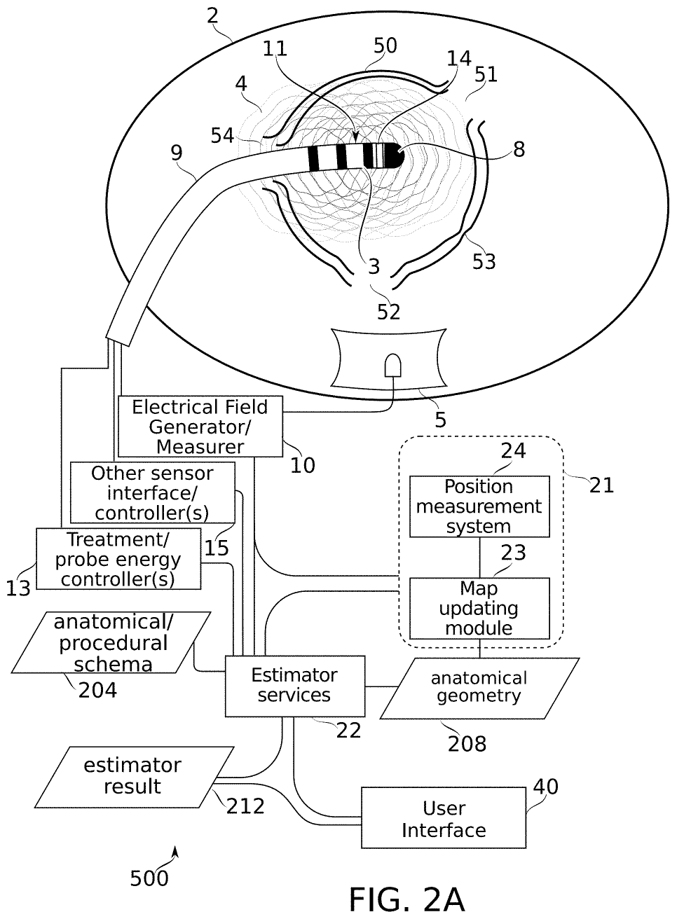

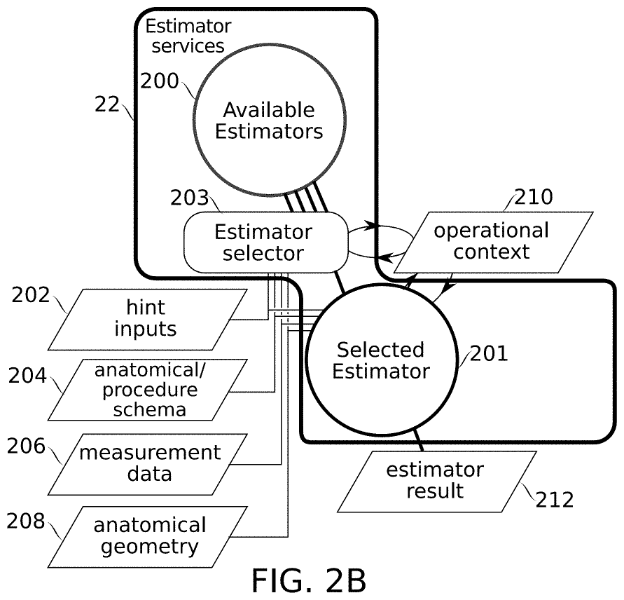

In overview, operational context 210 comprises:
system settings of system 500 (FIG. 2A), for example, how electrical field generating electrodes are positioned and operated, and/or how treatments are set to be delivered;
positioning and/or procedural state (e.g., activation state) of an intrabody probe;
monitoring and control system supporting the intrabody probe; and/or
the state of the patient undergoing the procedure.

For example, operational context 210 corresponds, in some embodiments, to the state of a system 500 such as the one described in relation to FIG. 2A, herein.

More particularly, operational context 210 may include data describing:
What procedure (e.g., a cardiac intervention procedure, for example a procedure to treat atrial fibrillation by ablation) is being performed,
Where elements of the catheter probe system and patient anatomy are in relation to each other (in particular, what parts of the patient anatomy are in the vicinity of the probe, that is, positioned so that the probe has both proximity and at least potential access to them),
In what condition those elements are, and/or
What stage the procedure has reached.

In some embodiments, a system configured to carry out the method of FIG. 1A tracks operational context 210 continually during a procedure. Tracking may be based on progress through a procedure schema 204B, for example. In an example of such an embodiment, a system may detect entry of a catheter into the vicinity (e.g., the lumen) of a right atrium, and based on this set the operational context 210 to comprise readiness to perform a transseptal crossing using the catheter.

Optionally, operational context 210 is set, at least in part, by explicit indications from a system operator (e.g., a physician). For example, when the operator is ready to begin a transseptal penetration, the operator optionally issues a command to the system to enter a transseptal penetration mode, which sets the new operational context 210 accordingly.

Measurement Data

In overview, measurement data 206 comprises any available data which relates to the procedure underway.

In some embodiments, measurement data 206 relates to tracked positions (for example, electrically, magnetically and/or ultrasonically tracked positions) of a catheter probe, and/or measurements made using sensors (e.g., force sensors and/or temperature sensors) and/or electrodes carried by the probe. In some embodiments, the catheter probe comprises catheter probe 11, for example as described in relation to FIG. 2A, herein.

Electrical measurements may comprise, for example, voltage measurements in response to currents introduced through the same probe electrodes and/or different electrodes, such as other internally introduced and/or body surface electrodes. Optionally, electrical measurements comprise measurements of endogenous electrical activity of nearby tissue. Optionally, measurement data 206 comprise data related to treatment activation and/or use of probing energies such as by heating, cooling, injecting, touching (optionally including measurement of contact and/or contact forces), irradiating, or otherwise interacting with nearby tissue.

In some embodiments, measurement data 206 comprise any other data acquired and/or entered coordinate with operations of the procedure, including patient data (e.g., patient medical history, and/or vital statistics), patient monitoring data (e.g. heart rate, temperature, and/or respiratory rate), and/or previously or concurrently acquired imaging data (CT, MRI, nuclear, and/or X-ray images, for example).

In some embodiments, recorded data is of location of a probe. The location may be recorded as absolute position and/or relative position. Optionally, location is recorded with respect to any suitable number of dimensions. For example spatial dimensions of a three-axis coordinate system may be recorded. Optionally, spatial dimensions are encoded indirectly, e.g., as position along a voltage gradient. Any number of voltage gradients may be used, for example, voltage gradients generated at different frequencies between a multiplicity of electrodes. Additionally or alternatively, time may be introduced as a dimension: linearly (elapsed time, for example), or cyclically (heartbeat phase and/or respiratory phase, for example). Optionally, location is recorded with respect to one or more functional domains acting as a “tag” or signature of the location, for example impedance measurements. Optionally, properties other than location which may vary as a function of tissue environment are used as tags; for example, any of the properties listed in the next paragraph.

In some embodiments, recorded data is of local tissue properties; for example: molecular structure (e.g., directions of molecular fibers, membrane integrity, and/or relative concentrations of molecular types such as fats and/or proteins), IR reflectance, HoYag laser reflection, endocardial and/or other electrical activity, pH, and/or ion concentration. Optionally electrical measurements related to exogenously created electrical navigation fields, local electrical impedance, and/or local electrical reactance are treated as “local tissue properties”.

In some embodiments, recorded data is of tissue change with respect to some variable. For example, tissue change may be a change of a tissue property as a function of: probe pressure, heating, cooling, time per se, heartbeat phase, respiratory cycle, heart rate, defibrillation, and/or delivery of energy. Optionally, the tissue change is monitored electrically from a probe electrode: for example as the tissue change affects local influences on impedance at one or more frequencies, the tissue change may be monitored by monitoring the local impedance at the tissue. Optionally, the monitored tissue change directly related to the monitored variable (for example, tissue heating/cooling may be monitored by monitoring the temperature).

Anatomical Schema

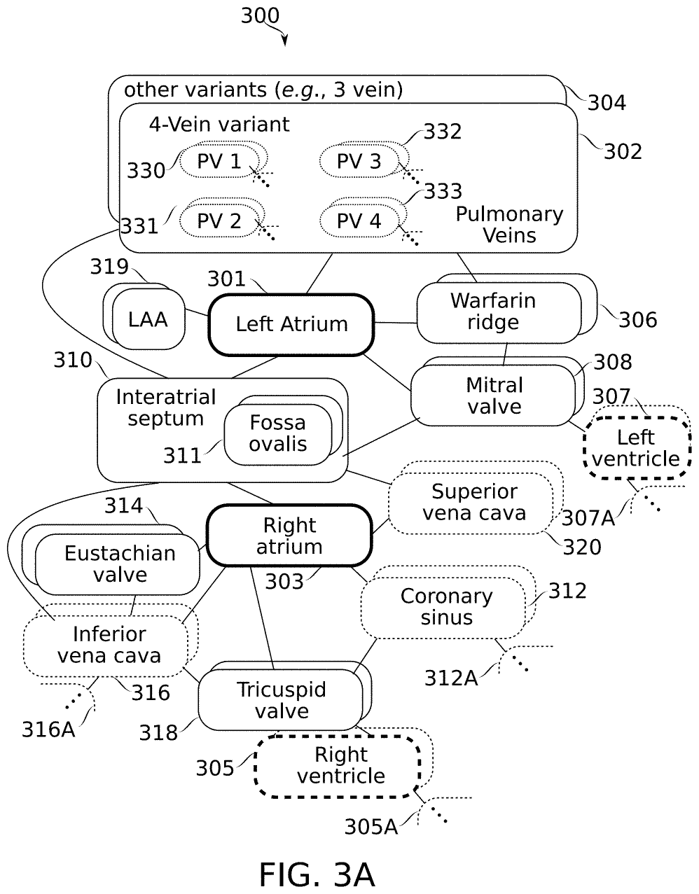

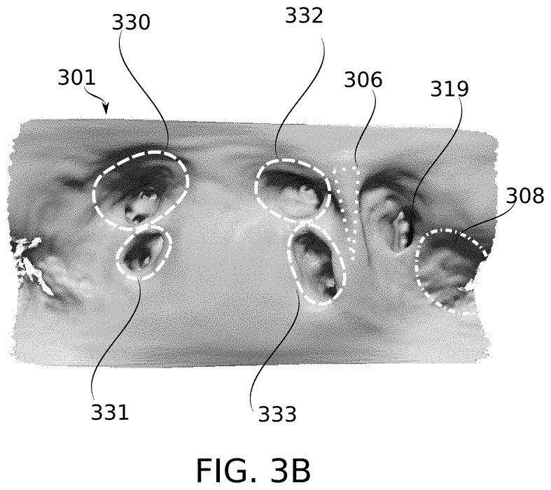

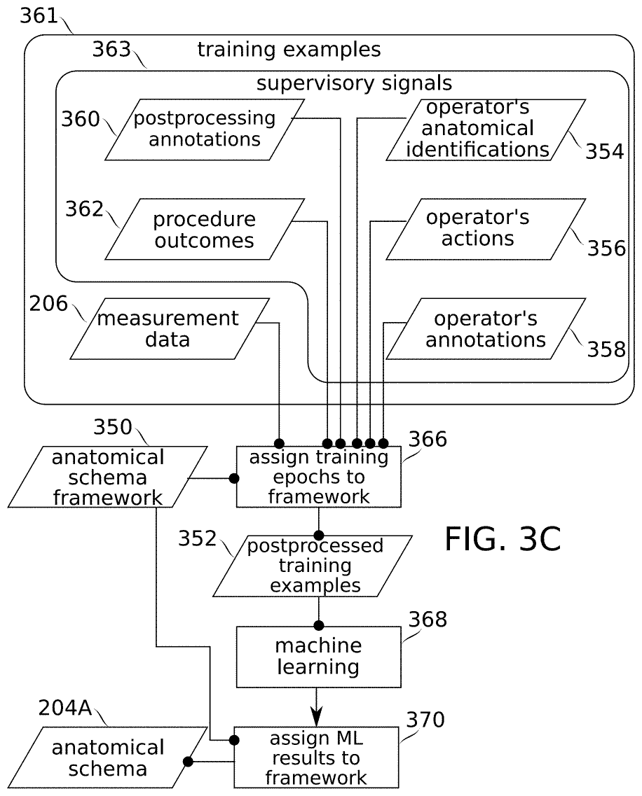

In overview, an anatomical schema 204A comprises a data structure or collection of data structures defining rules. The rules relate a plurality of anatomical identities to one another (e.g., so that knowing the anatomical identity of first structure gives information, under the rules, about what other anatomical structures are nearby that first structure), and/or relate characteristics of measurement data 206 to particular anatomical identities, at least within the operational context (which may be an anatomical location and/or a procedure phase) where the rule is relevant. Optionally, an anatomical schema 204A (or rule thereof) is defined for a particular operational context 210 and/or as a function of operational context 210. A schematic representation of an anatomical schema is described, for example, in relation to FIG. 3A.

Herein, the term “rule” is used to describe any function, equation, table, model, machine learning output, or other expression which can be evaluated together with some input to produce a result; for example, a number, truth value, a selection from a range of options, a deductive conclusion, an inductive conclusion, and/or a statistical likelihood. Moreover, to be explicit: although certain types of machine learning results are sometimes described as expressing input/output associations without embodying distinct rules, herein such a machine learning result may nevertheless be considered, in and of itself, to embody at least a rule: that is, the rule of the expressed association that the machine learning result itself embodies.

As a partial example, again in the context of a procedure comprising transseptal penetration:
an “interatrial septum” is optionally defined in an anatomical schema as comprising: a “fossa ovalis”
   wherein the fossa ovalis surrounded by regions (e.g., tracked positions in contact with wall tissue) which are “not fossa ovalis”; and
wherein a rule distinguishing between the “fossa ovalis” and “not fossa ovalis” operates on the basis of:
   impedance measurement differences that correlate with wall thickness (the fossa ovalis being found over a thinner portion of the wall), and
   optionally also on the basis of where the thinning is located (i.e., in a central region of the interatrial septum).

While the above description is presented in natural language for the sake of description, it should be understood that in some embodiments, a representation of an anatomical schema 204A for use in automatic processing is encoded in a suitable machine-readable format. Encoding optionally uses, for example, XML (e.g. according to a purpose-designed XML schema), JSON or another computer language-derived data structure description, a numerically encoded (“binary”) format, weights for a neural network, another format suitable for encoding machine-learning derived algorithms, or in any other suitable format.

Relationships among regions of different anatomical identity which may be encoded as rules (explicitly by coding and/or implicitly by machine learning) in an anatomical schema 204A may include, for example, any one or more of the following, and/or their opposites as applicable:
Containing, being contained, comprising, or another relationship of “composition”;
Adjacency, overlap, relative orientation, opposition (positions opposite one another, e.g., within a lumen), relative distance, relative size or another relationship of spatial position and/or extent;
Co-occurrence, mutual exclusivity, and/or likelihood of either; and/or
Property correlations and/or relative values (e.g., co-variation and/or consistent relative magnitudes, e.g., of lumen size, wall thickness, reactivity to stimulation, and/or susceptibility to edema).

In some embodiments, an anatomical schema 204A includes alternative rules which allow the anatomical schema 204A to encompass certain types of anatomical variability in a population. For example, in a normal population, potentially 75% of the population will have a fossa ovalis (a depression in the right atrium of the heart, at the level of the wall between right and left atrium, which is the remnant of a thin fibrous sheet that covered the foramen ovale during fetal development), and 25% of subjects will have a PFO (patent foramen ovale; that is, a full opening in the interatrial septum dividing the right and left atria, instead of a mere reduction in wall thickness). An anatomical schema may include both characteristics of fossa ovalis and PFO. Other well-known variations in cardiac anatomy include but are not limited to:
Unusual persistence (and/or size) of the Eustachian valve which is normally only functional in fetal circulation,
Numbers of pulmonary veins leading to the left atrium other than four (three, for example), and
A relatively pronounced ridge between the pulmonary veins and the left atrial appendage (sometimes called a “warfarin ridge” for its resemblance in some diagnostic results to a thrombus, which may lead incorrectly to treatment with clot-thinning drugs).

It is noted that the rules of an anatomical schema 204A do not necessarily operate the basis of precise descriptions of anatomical geometry (e.g., do not necessarily require reconstructions of tissue surfaces). They may do so, in some embodiments. In other embodiments, the rules of an anatomical schema 204A definitely do not operate the basis of reconstructed tissue surfaces. Optionally, the rules do not operate on image data. In some embodiments, anatomical schema 204A includes distributions of anatomical geometries, for example, data pertaining to the frequency at which certain distances between two anatomical landmarks may appear.

Even if precise position data is available (for example, based on position tracking of a probe), use of comparison rules established by an anatomical schema 204A optionally ignores some or all of this precision. For example, it may not be relevant to a rule to know just how close to the center of the interatrial septum a candidate position for a fossa ovalis is, so long as, for example, it can be determined that there are a substantial number of distinct positions between it and regions with properties defining the outer boundaries of the interatrial septum.

Procedure Schema

Optionally, the method of FIG. 1A continues at block 132. The main difference from block 130 is that the selection operation of block 132 selects an action (output as selected action 212B), rather than an anatomically identified target as such.

Inputs to block 132, in some embodiments, include operational context 210, measurement data 206, and procedure schema 204B. Optionally anatomical schema 204A (e.g., the anatomical schema 204A used at block 130) is also included as input. However, procedure schema 204B may itself be considered as a particular type of anatomical schema 204A (and indeed, is optionally implemented as one), in which the rules that relate and characterize different anatomical identities are also provided with indications (derived from rules applied to inputs) of what actions should be performed on regions having those anatomical identities in the context of a particular procedure and/or phase of a procedure. Herein discussions of aspects of an anatomical schema 204A should be understood to apply also to a procedure schema 204B, except as otherwise noted.

For example, the action associated with a PFO in a procedure schema 204B may simply be to pass a catheter probe through the open hole, while the action associated with a closed fossa ovalis may be to penetrate it by needle and/or the use of an ablation probe. In this case, the particulars of the selected target 212A (open or closed hole) interact with the operational context 210 (transseptal penetration) to select alternate options encoded by the procedure schema 204B. In some embodiments, the selected action 212B is subject to more granular control—for example, if an ablation-assisted transseptal crossing is selected, the selected action 212B optionally comprises specification of ablation parameters to be used, which may vary, for example, based on the measured and/or anticipated thickness of the fossa ovalis.

In some embodiments, selected action 212B is provided as an indication to an operator which may be treated by the operator as an option, suggestion, and/or recommendation. In some embodiments, selected action 212B is automatically used by a system to set parameters for the next operation (optionally while maintaining an available option for the operator to override the parameters. In some embodiments, selected action 212B is begun automatically by the system as soon as some criterion is met—for example, ablation is optionally begun (e.g., with prior operator permission) as soon as the system reaches some predetermined degree of confidence that the catheter probe is currently in contact with the true fossa ovalis.

Target/Action Selection within a Procedure

Reference is now made to FIG. 1B, which is a schematic flowchart of the use the method of FIG. 1A within the context of a procedure, according to some embodiments of the present disclosure.

At block 102, in some embodiments, a determination is made as to whether there is currently available a valid context, based on which further processing can proceed. If yes, the flowchart continues at block 106. Otherwise, flow continues to block 104, at which a context is set.

FIG. 1B introduces an optional distinction between two aspects of operational context 210—anatomical context 210A and procedural context 210B. There need not be a sharp distinction implemented between these two. At least for purposes of description, however, anatomical context 210A may be understood to comprise information describing the “where” of the current context—for example, where a catheter probe is located, and/or where a current target (e.g., for ablation) of the catheter is located. Procedural context 210B describes the “what” of the current context, for example, what a goal of a current phase of a procedure is (or other features of the current procedure phase). Optionally the two types of context are intermingled in their used and/or definition. Optionally, only one of the context types is used and/or defined. For example, in a defined procedural context, all information about anatomical context is optionally subservient to “what to do next”. An interatrial septum, for example, is optionally treated as only relevant during the phase of the procedure where it becomes a target for the action of crossing it. This perspective optionally allows taking a focused approach to defining “context”, which has the potential advantage of controlling complexity. On the other hand, the approach can be brittle, since if a procedure leaves the main path of the procedure (e.g., by accident), there may not be a well-defined way to guide a return.

“Setting” a context 210A and/or 210B is optionally manual, automatic, or a blend of the two. An example of manual context setting is to simply have a user inform a system, e.g., that the procedure is now in some particular phase (related to procedural context 210B), a catheter is now in some particular place (related to anatomical context 210A), and/or a particular goal of the current phase has now been reached (again, more related to procedural context 210B). Then the system can set a new context, based on that input. The input can be, for example, via user interface 40, for example as described in relation to FIG. 2A.

In an example of automatic context setting, a system is optionally configured to recognize a context based on automatically acquired measurement data 206 (for example, but not necessarily, in conjunction with the use of one or more rules of an anatomical schema 204A). For example, after sufficient exploration of a right atrium (without necessarily knowing that it is a right atrium), a system optionally has available to it sufficient information to constrain a catheter probe as being within a chamber of a certain minimum size, and connected to two large, oppositely situated blood vessels. Optionally, a rule of an anatomical schema 204A is defined so that these characteristics uniquely (or at least probabilistically) indicate that the probe is indeed located within a right atrium. Optionally (for example, based on the location of the probe, its entry point, and the positions of the two blood vessels), the system is also able to determine in what direction from the probe lay other potential target features of the right atrium. Such features could be, for example, the interatrial septum, the opening into the coronary sinus, and/or the plane of the tricuspid valve. In some embodiments, manually provided “seed” context is used to orient the system, after which acquired data are matched to suitable anatomical identities defined by application of rules of the anatomical schema 204A based on sequential encounters during a procedure. For example, catheters passing in from the jugular vein or the femoral vein should enter the heart itself in different locations, so that data indicating entry to a heart chamber would be interpreted differently in each case.

At block 106, in some embodiments, a context-appropriate target estimator is selected. Generally, an “estimator” is a rule for calculating an estimate of a given quantity based on observed data. An estimator may be, for example, a rule defined by an anatomical schema 204A. A target estimator, more specifically, is an estimator in which the “given quantity” corresponds to the identification and/or detection of an anatomical target, the suggestion of an action to be performed upon such a target, and/or is an estimation of at least one of the target's properties. The estimated “given quantity” may be, for example:
Boolean (target is/is not a certain structure, e.g., a fossa ovalis).
A choice from a multiplicity of options (for example, target is one of N possible structures, target is in one of N possible states; e.g., target is a fossa ovalis, a patent foramen ovale, or another position on a septal wall).
A specification on a range (for example, target has a certain wall thickness; target has a certain probability of being a fossa ovalis rather than a septum).
A combination of a plurality of given sub-quantities.

A target estimator is “context-appropriate” insofar as the rule it comprises is appropriate for the current situation when block 106 is entered. A target estimator for identification of a fossa ovalis, for example, is potentially context relevant when seeking a crossing location for a catheter tip between a right atrium and a left atrium. The same target estimator is not context-appropriate while the catheter tip remains in the vena cava, or after it has made the crossing.

Block 106 relates to the beginning of operations performed in block 130 of FIG. 1A. In some embodiments of the invention, the types of measurement data 206 used in target selection, as well as how that data is used, can be very different depending on the current context. From the right atrium, for example, an estimator for finding the entry to the coronary sinus should look for different characteristics than an estimator for finding the fossa ovalis. Knowing the current anatomical and procedural context 210A, 210B (in this case, it is procedural context 210B that is distinguishing) potentially allows the correct estimator to be selected.

At block 108, in some embodiments, data (that is, data corresponding to measurement data 206) is collected, for example as a catheter probe is moved around within the general vicinity of the target being sought. As data is collected, it is possible that the context will change (intentionally or by accident); so at block 110, context is periodically updated based on the same measurement data 206. At block 112, if the context is no longer valid for the current target estimator, flow returns to block 104, where a new context is set (or verified), and that part of the process begins again. Otherwise, at block 114, the estimator selected at block 106 is used in an attempt to estimate the current target and/or target-specific action (as appropriate).

The estimate attempt may or may not succeed; for example, there may be insufficient data to make a good early estimate. At block 116, a determination is made as to whether the estimate result should be treated as valid. If not, more data is collected at block 108. Otherwise, the flowchart proceeds to block 118, at which an action on a target is made. Either the action, the target, or both may be specified from the results of block 114 (with the operator tacitly responsible for accepting the specification, and supplying whatever part may be missing).

At block 120, a determination is made as to whether the procedure has completed or not. If not, flow returns to block 104, at which a new context is potentially set. Otherwise, the flowchart ends.

### Examples of System Embodiments

System Overview

Reference is now made to FIG. 2A, which schematically illustrates a system 500 for use in performing the methods of FIGS. 1A-1B, including a schematic representation of a patient body 2, according to some embodiments of the present disclosure.

At the core of system 500 (for purposes of the present description) is a block representing estimator services 22. This block is described in more detail in relation to FIG. 2B. The estimator services are optionally implemented by a computer processor programmed to accept inputs and provide outputs as described, for example, in relation to FIGS. 1A-1B and/or 2B. In some embodiments, inputs to estimator services 22 comprise anatomical/procedure schema 204 (which may comprise one or both of anatomical schema 204A, and procedure schema 204B). The other connections leading into estimate services 22 comprise examples of various sources of measurement data 206.

As an input to estimator services 22, user interface 40 may be used to set context and provide other user-generated selection data, for example as described in relation to block 104 of FIG. 1B. User interface 40 also functions as an output for, among other functions the system may require, indications provided as estimator results 212 of estimator services 22 (e.g., selected target 212A and/or selected action 212B of FIG. 1A).

The remaining inputs shown to estimator services 22 emphasize the role of an intrabody probe 11 of a catheter 9 in sensing various parameters for use in the operations of estimator services 22. It is to be understood that other input sources are optionally used; for example, as described in relation to block 206 of FIG. 1A. Electrical field generator/measurer 10 is provided as a general purpose block covering all electrical sensing functions. Optionally, it is implemented as a plurality of sub-modules. In some embodiments, a major function of electrical field generator/measurer is to generate and sense electrical fields 4 for use in navigation, for example, using pairs (and/or other configurations of body surface electrodes 5 (only one electrode is shown in the schematic drawing). In some embodiments, navigation comprises detecting voltages using electrodes 3 of probe 11 as they move through a plurality of crossed (e.g., approximately orthogonal) time-varying voltage gradients. Each gradient is distinguished, for example, on the basis of frequency. Optionally, the crossed fields are treated as coordinate axes, optionally transformed as necessary to produce 3-D spatial coordinates. While body surface electrode-generated fields are used in FIG. 2A as an example, fields used for electrical navigation are optionally produced from other sources; for example, from intrabody electrodes located near a body cavity 50 to be navigated (e.g., in the coronary sinus for coronary navigation requirements). Optionally, the electrodes of probe 11 itself are used to both produce and sense electrical fields, and the sensed voltages treated more as “tags” than as coordinates on coordinate axes.

In some embodiments, measurements made by electrical field generator/measurer 10 are relayed to position services module 21 (optionally implemented as software running on a processor). By whatever method is appropriate to the configuration of the system, position measurement system 24 converts the voltage measurements from the probe into probe positions, while map updating module 23 uses these positions to generate a map of the body cavities which probe 11 navigates. Over the course of a procedure, and in particular for regions which probe 11 visits exhaustively, there may be a highly detailed map generated. However, this condition of dense visitation potentially does not hold (and/or holds at the cost of inconvenience and procedure delay) for all regions, and anyway there is potentially a significant period of time that passes before high-resolution map is available. Nevertheless, the positions and maps created and/or maintained by position services module 21 are provided as inputs to estimator service module 22, in some embodiments, as a source of data on which target and/or action estimators operator. Optionally, but not necessarily, this data is provided in the form of a current best estimate of anatomical geometry 208. Optionally, anatomical geometry 208 is estimated based on results of a prior catheter procedure. Optionally, anatomical geometry 208 is estimated at least in part and/or initially based on currently or previously acquired imaging data, for example, imaging by CT, MRI, NM, ultrasound, X-ray, or another imaging technique. Optionally, anatomical geometry is estimated at least in part and/or initially based on anatomical atlas information.

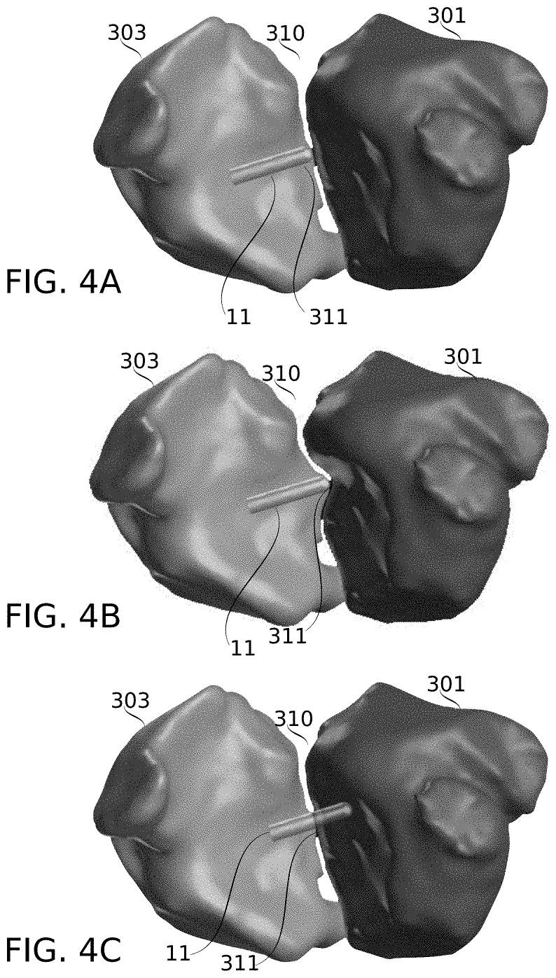

The data produced by electrical field generator/measurer 10 optionally include data other than that which serves as a direct basis for measured spatial position or navigation. In particular, electrodes 3 may be operated to obtain data influenced by the local electrical environment of tissue, for example dielectric property data; or more generally, differences in impedance or other basic electrical properties as a function of local tissue environment. Two types of anatomical features which are particularly distinguishable from such data are approaches of an electrode probe 11 to tissue walls, and the relative thickness of those walls as electrode probe 11 moves along them. This allows distinguishing, for example, more confined cavities (e.g., passages into/out of body cavity apertures 51, 52, 54) from more open cavities, and thicker walls from thinner ones (e.g., thin wall feature 53). Such electrical properties and their uses are described in connection with embodiments of specific applications described herein, for example, in relation to FIGS. 4A-4C, 5, 6, 7A-7B, 8, 9A-9D, 10A-10B and 11 herein.

In some embodiments, one or more non-electrode sensors 14 is optionally provided, either as an integral part of probe 11 (as shown), or as part of an auxiliary probe used with it. Such a sensor may comprise, for example, a force and/or temperature sensor. Data from such sensors is optionally collected by other sensor interface controller(s) 15, and provided to estimator services 22 as another form of input.

In some embodiments of the invention, probe 11 comprises one or more elements 8 supporting one or more treatment modalities. Examples include elements for cryoablation (balloon and fluid conduits, for example), one or more RF ablation electrodes, injectable substances and their injection means (needle), or another treatment modality. In some embodiments, details of the operation of treatment probe energy controller(s) 13 are provided to estimator services 22, for example to assist in the evaluation of changes produced as a result of manipulation via element 8. Optionally, treatment parameters' under the control of controller 13 are controlled and/or suggested based on outputs from estimator services 22 (for example, in embodiments where an output of estimator services 22 comprises parameters of a selected action 212B).

Estimator Services

Reference is now made to FIG. 2B, which schematically represents inputs and operations of an estimator services module 22, according to some embodiments of the present disclosure.

In some embodiments, external inputs to estimator service module 22 include hint inputs 202, anatomical/procedure schema 204, measurement data 206, and/or anatomical geometry 208.

In some embodiments, hint inputs 202 comprise one or more forms of non-measurement data which are used by estimator services 22 in setting context which may help in selecting an estimator (for example as described in relation to block 104 of FIG. 1B), and/or provide information used by an estimator (e.g., selected estimator 201) to produce an estimator result 212. In some embodiments the hint inputs comprise explicitly provided inputs from an operator, for example, inputs specifying a location of probe 11, a port of entry of probe 11, which probe 11 of an optional plurality of probes is being used, operational phase of a current procedure, a selection among potential anatomical variants, or another input.

In some embodiments, hint inputs 202 comprise information implicit to the choice of system configuration and/or procedure. For example, estimators which rely on electrical field navigation-type position inputs are normally unavailable for selection by estimator selector 203 if electrical field navigation is not being used. Hints can also include, for example, specification of the point of initial access of a catheter to a body (e.g., femoral vein or jugular vein) and/or details of anatomy (for example, the presence of variant anatomy structures) which may be known from previous data such as prior catheter and/or imaging procedures.

Anatomical/procedure schema 204, in some embodiments, comprises one or more rule-defining data structures configured as described, for example, in relation to FIG. 1A, and/or as described in relation to the schematic example of FIG. 3A.

Measurement data 206, in some embodiments, comprises data from one or more sources of measurements, for example one of the sources listed in relation to block 206 of FIG. 1A, and/or described in relation to the various data collecting elements of FIG. 2A.

Anatomical geometry 208, in some embodiments, comprises a current estimate of patient anatomy in a region of interest, for example as described in relation to block 204 of FIG. 2A.

In some embodiments, of estimator services 22 comprises two main operations: (1) selection of a selected estimator 201 by an estimator selector module 203 from among a pool of available selectors 200, and (2) use of the selected estimator 201 to produce an estimator result 212, based on currently available inputs. These operations are described, for example, in relation to FIG. 1B. It is noted that in some embodiments there is also maintained by the estimator services 22 an operational context 210, comprising one or both of a current anatomical context and a procedural context.

### Examples of Anatomical Schema

Reference is now made to FIG. 3A, which schematically represents selected anatomical relationships encoded by rules of an anatomical schema 204A, according to some embodiments of the present disclosure. Reference is also made to FIG. 3B, which illustrates some of the left atrium 301 features mentioned in FIG. 3A in an “unwrapped” view of the left atrium, according to some embodiments of the present disclosure.

The portion of the anatomical schema 204A illustrated in FIG. 3A emphasizes relationships among regions of different anatomical identities that relate to the atrial chambers of the heart (that is, rules governing aspects of their spatial relationships to one another). It is to be understood that the diagram of FIG. 3A is a visual representation of logical relationships which would normally be otherwise encoded (e.g., as XML, JSON, and/or a binary format), for example as described in relation to block 204A of FIG. 1A. Elements of the diagram illustrate examples of features mentioned in that description, for example relationships of position and composition.

In some embodiments, an anatomical schema 204A may include coverage of all or any suitable fragment of the anatomical structures shown in FIG. 3A, and optionally different or additional anatomical structures. Potential advantages of a relatively more complete anatomical schema 204A include coverage of more situations (e.g., more navigation regions, different available data, more types of anatomical variants), and/or increased reliability of automatic inferences made using rules of the schema (e.g., because more lines of evidence potentially converge to confirm an identification of a target). A more complete anatomical schema 204A may also be useful for uninterrupted control and/or monitoring of the flow of operations throughout a larger section of the overall procedure, for example for support of multi-chamber operations. In contrast, a relatively fragmented anatomical schema may still be of value for providing assistance during particularly difficult and/or error-prone phases of a procedure. For example, a fragmented anatomical schema may comprise just rules for identifying a fossa ovalis target within an interatrial septum, assuming prior localization of the interatrial septum. As previously noted, a procedure schema 204B is optionally implemented as a narrowly defined anatomical schema, wherein each anatomical identity in the schema is provided with information particularly tailored to progressing the procedure from one phase to the next. Optionally, identities shown in FIG. 3A as “anatomical identities” are recast as “procedural identities”, focusing on phases of navigation and/or intervention such as “enter right atrium”, “cross the IAS”, “ablate” and the like—in this case, anatomical identities are optionally entities subservient to the exigencies of each sequential operational phase of the procedure.

For brevity, in the descriptions of FIG. 3A that follow, schema entries comprising collections of rules for particular anatomical structures (anatomical identities) are referred to by common names for those anatomical structures. However, it should be understood that such references with respect to FIG. 3A are actually to the portion of the anatomical schema data structure that pertains to the actual anatomical structure mentioned, not the anatomical structure itself.

Beginning with schema entry for the right atrium 303, FIG. 3A shows that the right atrium 303 is directly connected to typical and/or variant right atrium features such as tricuspid valve 318 (leading to the right ventricle 305), inferior vena cava 316, coronary sinus 312, superior vena cava 320, interatrial septum 301, and Eustachian valve 314. This list of connected elements is not necessarily exhaustive, and any given implementation of an anatomical schema optionally adds or removes schema entries as appropriate for the particular procedure(s) which are to be supported. Several of the schema entries of FIG. 3A are indicated with doubled overlapping enclosures. This is to indicate the optional presence of variant anatomies, the detection and encoding of which is described herein with respect to some selected examples. Another convention of FIG. 3A is the use of partial boxes to indicate the optional inclusion in some embodiments of additional schema entries not shown in FIG. 3A, for example unnamed features 305A, 312A, 307A, and 316A, connected their correspondingly numbered (without the terminal “A”) anchoring schema entries.

One example of an anatomical variant is Eustachian valve 314, a valve of the inferior vena cava (IVC) which is large in the fetal stage, and plays an important role in fetal circulation as it directs oxygenated blood from the maternal placenta directly across the patent foramen ovale into the left atrium thereby reaching the left ventricle (avoiding the lungs) and being pumped, e.g., to the brain. In some embodiments, the maintained and/or enlarged presence of this valve in an adult patient is associated with increased the risk of right to left paradoxical shunt of emboli across the PFO (stroke). In some embodiments of an anatomical schema, a criterion for noting the presence of an enlarged Eustachian valve comprises a finding of interference with movements and/or positioning of a probe 11 in the region of the IVC (particularly compared to the superior vena cava, SVC). In some embodiments, such a criterion comprises a finding of otherwise unexpected fluctuations in measurements (e.g., of impedance) consistent with contact with a wall or flap, in a place where an anatomy would ordinarily be expected to be free of such a wall or flap. Meeting one or both of these criteria in a certain location optionally not only sets that location “Eustachian valve”, but also helps to identify a nearby region having, for example, impedance and/or navigationally restricting features of a blood vessel inlet as being more specifically the inlet to the right atrium of the IVC. This in turn allows the deductive inference that a second such blood vessel inlet is the SVC. From this the orientation of the right atrium is now known, allowing localization of the direction in which the interatrial septum 310 lies, and, at least along the IVC/SVC axis, something about its extent. Similarly, the general position of features such as the coronary sinus and tricuspid valve can be automatically deduced (crossing the tricuspid valve, for example, is optionally noted from changes in intra-cardiac ECG), and any “sinus like” or “valve like” features in those positions assigned to be actually the appropriate feature with a high degree of confidence.

Similar chains of deduction can be built up from different starting points and/or hints. For example, if it is known that the catheter procedure began from a femoral vein, then the IVC/SVC distinction can be inferred based on the identity of the vein-like aperture the catheter first enters when entering the right atrium. Entry to the right atrium itself may be detected (after entry) by such features as how many and/or what relative size of apertures lead from it (once it is mapped to sufficient completeness), and/or how far a probe can move from the entry point in one or more directions before encountering a wall. The wall encounter may be identified by a characteristic impedance change. Additionally or alternatively, the probe position may be identified as being in the right atrium by an impedance reading which indicates that all tissue walls are far away from the probe (e.g., more than a threshold distance from, and/or not showing impedance measurements characteristic of proximity to), identifying a pronounced heartbeat cycle-dependent fluctuation (e.g., by corresponding fluctuations in impedance or other electrical readings), detection of electrical impulses propagating through the walls of the chamber, and/or another distinguishing property of the right atrial chamber environment measurable by a probe situated therein. Any or all of these types of property-based indications and/or logical deductions are optionally provided as explicitly encoded features of an anatomical schema 204A. However, in some embodiments, some or all of these aspects are found and encoded implicitly, for example based on supervised machine learning techniques, for example as described in relation to FIG. 3C.

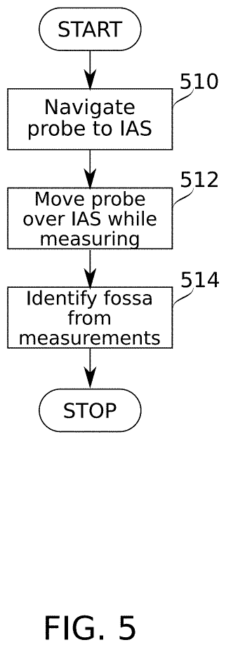

Another situation for which an anatomical schema may provide guidance is in the location of a fossa ovalis (or PFO), for example as described in relation to FIG. 5, herein. With respect to the structure of the anatomical schema, it is noted for now that the fossa ovalis 311 is optionally encoded as an anatomical sub-identity of the interatrial septum 310. For purposes of locating the actual fossa ovalis, a search strategy optionally proceeds first by finding some part of the interatrial septum, and then by further search locating the fossa ovalis 311 itself.

Continuing from the schema entry for the fossa ovalis 311, the anatomical schema of FIG. 3A also comprise a schema entry for left atrium 301. Visual appearances of some of these features can be seen in an unwrapped view of the internal lumenal surface of a left atrium shown in FIG. 3B (in FIG. 3B, reference characters label anatomical features as such using the same numbering scheme applied to the anatomical names applied more narrowly to schema entries in the descriptions of FIG. 3A).

In addition to the fossa ovalis 311 and interatrial septum 310, left atrium 301 is also connected to several other features which line (or may line its interior lumenal wall, including the left atrial appendage (LAA) 319, the pulmonary veins 302, the so-called (and optionally present in variant forms of various sizes) warfarin ridge 306, and the mitral valve 308 (which leads to the left ventricle 307, which has not been detailed in the figure).

Of particular interest as an example is the potentially variant anatomy of the pulmonary veins, which can potentially be present as the canonical 4-vein variant (pulmonary veins (PV) 330, 331, 332, 333), or in another variant form 304 such as a three-vein variant. In some embodiments, an anatomical schema is adapted to automatically select from among possible variants based on numbers of aperture features actually encountered, and/or based on where aperture features are encountered (for example, encountering an unusually large ostium in a position intermediate to the canonical four-vein positions of two PVs is optionally treated as evidence that the three-vein anatomical variant of the anatomical schema should be used.

Thus, each schema entry for a certain anatomical identity is optionally locatable based on at least one of the following types of information:
How it is positioned and/or oriented with respect to other identified anatomy parts;
How it is positioned and/or oriented with respect to a probe being used in the procedure;
What sorts of properties it is expected to have (even if those properties as such are only partially identifying, they may be used together with information about the overall anatomical context to form a full positive identification). Examples include properties of impedance measurements (e.g., considered singly, differentially, and/or as a function of frequency, time and/or position), or any other property for example as described in relation to FIG. 1A.
What sorts of properties help to distinguish relevant anatomical variants from one another.

In some embodiments, as different regions of an anatomy are automatically provided with anatomical identities, a system indicates these identities to a user through user interface 40. Optionally anatomical identities (previously and/or currently provided as selected target 212A, for example) are associated with a degree of confidence, which potentially may be increased by the acquisition of additional data. Optionally, indications can be manually set by system operators. Optionally, automatically determined indications can be edited and/or overridden by system operators. Manual identification input may be used, for example, as supervised results paired with training data collected for use in machine learning of associations that produce target and/or action estimator results 212 from input measurement data 206.

In some embodiments, anatomical identities are shown on user interface 40 as tags, for example, character abbreviation tags, colored spheres (with associated dictionary), fully colored and/or textured regions of anatomical surfaces (e.g. heart chamber and/or vascular wall), shading effects to simulate surface features (e.g., bump mapping to highlight an identified region of a fossa ovalis), and/or special lighting effects applied to a rendered view approximating the anatomical geometry. For example, lighting may be simulated within the PVs and/or atrial-ventricular valve planes to mimic the color Doppler scheme according to direction of blood flow (e.g. blue-away, red-towards, or another convention). Optionally, tags that apply to hidden surfaces (for example, coronary sinus ostia) are visualized by, for example, changing the opacity with which an anatomical geometry is displayed, and/or applying a clipping plane to the display. Optionally, tag display effects are modulated to indicate confidence, for example, made more transparent, less saturated in color, differently textured, made more diffuse, or otherwise modified. Optionally, confidence is simply displayed as graphical indications like bars, dots, and/or numbers.

Actions (for example, selected action 210B) selected on (e.g. recommended for) a target region are optionally signaled by arrows, glowing and/or pulsing markers, or other signals. Certain types of actions are typically accompanied by changes in shape or position which can be inferred from non-imaging readings. For example, crossing of the fossa ovalis may be accompanied by characteristic “tenting” for example as described in relation to FIGS. 4A-4B. In some embodiments, these changes are simulated in a display for the user, wherein the simulation is synthesized on the basis of available non-imaging data.

Machine Learning Results Used with Anatomical Schema

Reference is now made to FIG. 3C, which is a schematic flowchart of the use of machine learning to establish at least some aspects of an anatomical schema 204A, according to some embodiments of the present disclosure.

Supervised machine learning comprises a family of techniques known in the art which are applicable to infer a function from a set of training examples (for example, training examples 361 of FIG. 3C). The training examples include pairs of inputs and their expected outputs (the outputs are provided as supervisory signals, for example supervisory signals 363 of FIG. 3C). The result of the machine learning is a function or other data structure which can be used to relate non-training inputs to new outputs in a way that (given a sufficient training set) follows input-output correlations found in the training examples.

In some embodiments, an anatomical schema 204A is built at least partially on the basis of machine learning results. In some embodiments, preparation of the training examples is performed on the basis of an anatomical schema framework which already includes many of the general features of the anatomical schema (e.g., which anatomical features are is adjoining to and/or contained by other features), but also has placeholder and/or empty functions for at least some of the functions that relate recorded measurement data to anatomical identities and/or recommended procedure actions. Machine learning results are optionally used to supply practical versions of these functions.

Measurement data 206 (described, for example, in relation to FIG. 1A) is largely what comprises the “input” side of the training examples, though the training example input may also be considered to include such things as patient history and other patient data, imaging data obtained outside of the procedure itself, and/or procedure design and parameters. As previously noted, in intervention procedures performed over catheter by indirect visualization, nearly all of the inputs and many outputs generated during a procedure are available in the same digital form originally available to practitioner.

Supervisory signals 363, in some embodiments, comprise at least one of:
operator's real-time or post-procedure corrected anatomical identifications 354 (optionally, these are corrected versions of the outputs of a previously available anatomical schema);
operator's actions 356 (what the operator actually did in a certain input context may be considered as an output reflecting the operator's particular expertise);
operator's other annotations 358 (for example, indications by an operator as to which particular parts of measurement data 206 were most relevant to anatomical identifications and/or actions);
post-processing annotations 360 (e.g., corrections of errors, linkage of outputs and inputs which are separated in the raw data such as ablation validations, annotations to interpret data and/or actions in terms defined by the anatomical schema framework 350); and/or
procedure outcomes 362 (e.g., results of a procedure which may only become known after the procedure itself is complete).

At block 366, in some embodiments, the training examples are optionally further processed so that appropriate epochs procedure during a procedure are assigned to be associated with the correct schema entries of the anatomical schema framework 350 (e.g., annotated so that they are associated with their correct anatomical and/or procedural context). The result of this, and any optional further post-processing such as normalization, is provided as post-processed training examples 352.

At block 368, the machine learning itself is performed, based on the post-processed training examples 352. Optionally, any suitable machine learning technique is used, for example, artificial neural network, back propagation, Bayesian statistics, case-based reasoning, decision tree learning, inductive logic programming, Gaussian process regression, group method of data handling, kernel estimators, learning classifier systems, multilinear subspace learning, naive Bayes classifier, maximum entropy classifier, conditional random field, nearest neighbor algorithm, probably approximately correct learning, symbolic machine learning algorithms, subsymbolic machine learning algorithms, support vector machines, minimum complexity machines, random forests, ensembles of classifiers, ordinal classification, data Pre-processing, statistical relational learning, and/or another machine learning technique.

At block 370, in some embodiments, the results of the machine learning at block 368 are assigned to the anatomical schema framework to produce an updated anatomical schema 204A.

Examples of Procedure Operations Used with Automatic Target/Action Selection

Interatrial Septum Crossing

Reference is now made to FIGS. 4A-4C, which schematically represent crossing by a catheter probe 11 from a right atrium 303 across an interatrial septum 310 to a left atrium 301 via a fossa ovalis 311, according to some embodiments of the present disclosure.

In FIG. 4A, probe 11 has found fossa ovalis 311, and is positioned against it. In FIG. 4B, probe 11 is pressing against fossa ovalis 311, causing “tenting” of the interatrial septum 310. In FIG. 4C, probe 11 has penetrated the fossa ovalis, releasing the tenting, and leaving probe 11 temporarily embedded half-way through the interatrial septum 310.

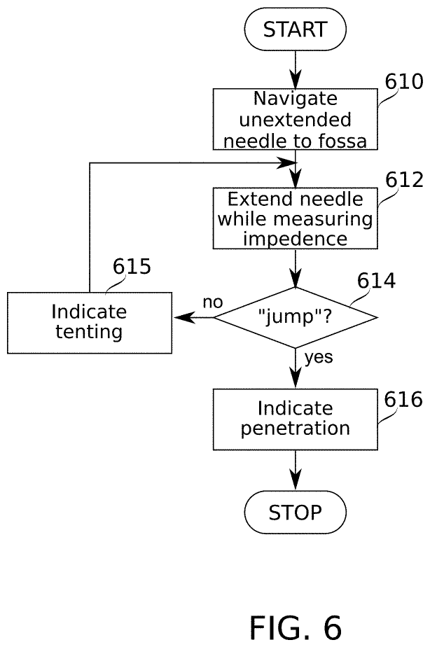

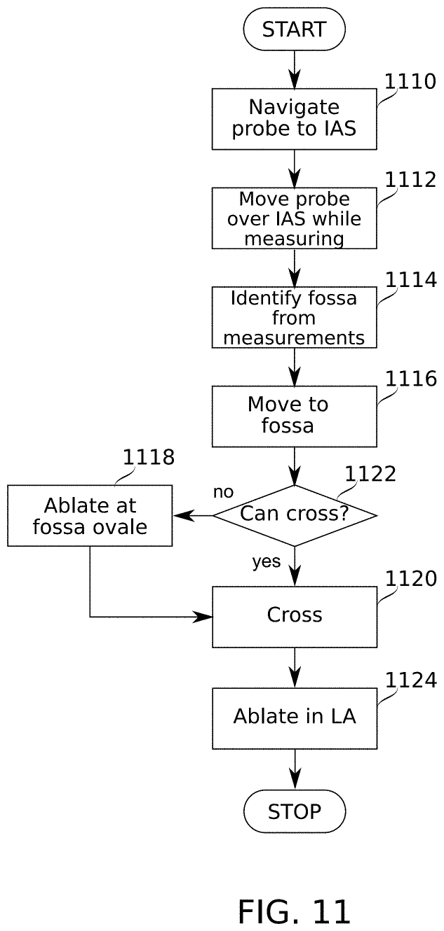

Different methods may be used to help encourage the crossing of a probe 11 as shown. Descriptions in relation to FIG. 11, herein, describe how ablation by a probe (e.g., RF ablation) may be used to assist crossing, potentially allowing a “single catheter” procedure for ablation to treat atrial fibrillation. Descriptions in relation to FIG. 6, herein, describe electrically monitored use of a needle to cross the interatrial septum.

Reference is now made to FIG. 5, which is a schematic flowchart describing a method of locating a fossa ovalis, according to some embodiments of the present disclosure.

At block 510, in some embodiments, a catheter probe 11 is navigated into contact with the interatrial septum (IAS). Discovery of the position of the IAS, for example with respect to the orientation of the IVC and SVC (optionally with assistance from the identification of the Eustachian valve) is provided in descriptions of FIG. 3A, in relation to an example of a schema entry for an interatrial septum 310. It is noted in particular that in some embodiments, a full right atrium map is optionally not generated—it is potentially sufficient to find the IAS, and scan it (by probe movements) in the general region where the fossa ovalis is expected to lie.

At block 512, in some embodiments, the catheter probe 11 is moved over the IAS while making dielectric measurements. It is generally not necessary to completely dielectrically map the IAS.

At block 514, in some embodiments, the fossa ovale or patent foramen ovale (PFO) (according to which is present) is identified.

In some embodiments, a fossa is identified based on a combination of voltage and/or impedance signals measured from probe electrodes 3, and geometrical considerations. The fossa is characteristically the thinnest zone in the septum (although in rare occasions it is lipomatous and thickened). A typical dielectric signature will vary from surrounding wall over a characteristics diameter of about 5-10 mm. Geometrically, the fossa is located about halfway between the SVC and IVC on the septal wall, between the septum primum and the septum secundum. The anatomical variant of an adult PFO may additionally or alternatively be identified as an open transseptal tract because the catheter probe simply crosses into the left atrium when it is pressed against the region of the PFO. It is noted that initially small 3-4 mm PFOs potentially increase in diameter with aging and can become stretched up to 7-10 mm (resembling a small to moderate atrial septal defect).

Monitored Needle Interatrial Septum Crossing

Reference is now made to FIG. 6, which is a schematic flowchart describing a method of crossing a fossa ovalis using an electrically monitored needle, according to some embodiments of the present disclosure.

Electrical monitoring of interatrial septum crossing using a Brackenrough needle and a NavX system (EnSite) has been described based on spatial position monitoring (Sumit Verma and Mark Borganelli, Real-Time, Three-Dimensional Localization of a Brockenbrough Needle during Transseptal Catheterization Using a Nonfluoroscopic Mapping System, J. Invasive Card., 18:7 (2006)). In some embodiments of the present disclosure, features of the electrical changes which occur during this penetration (not necessarily observations of position per se) are used to generate a visual representation of the procedure which evokes the “tenting” phenomenon which may be observed, e.g., under direct imaging visualization of a transseptal penetration.

The flowchart begins, and at block 610, in some embodiments, a catheter including a transseptal needle encased in a sheath is navigated to the region of a fossa ovalis. The needle itself (which is quite long, e.g., about 70-110 cm long, so that it may extend out of the body even with its tip inside the heart) can be used as a sensing electrode by electrically connecting it to, e.g., electrical field generator/measurer 10. Optionally, a proximal part of the needle is connected using an alligator clip through the pin-box to the system, converting it to a long, though insulated along its length, unipolar electrode.

In some embodiments, the transseptal needle itself is used to find the fossa ovalis, for example, as described in relation to FIG. 5. Optionally, the fossa ovalis is found separately from the action of crossing the fossa ovalis using the needle.

At block 612, in some embodiments, the needle is gradually extended from its sheath. The progress of the operation is optionally tracked by noting the changes in electrical signal as more and more of the needle is protruded from the electrically insulating sheath.

At block 614, in some embodiments, detection is made as to whether or not a sudden jump in electrical signal amplitude has occurred.

If not, optionally (at block 615), a display (e.g. on user interface 40) presents penetration progress to an operator by imitating the typical ‘tenting’ of the IAS before a successful puncture. Flow continues with a return to block 612.

Otherwise, at block 616, the jump is interpreted as a successful penetration. The “tenting” display is optionally returned to the IAS's resting position, but with the penetration need now shown crossing the IAS. The flowchart ends.

Monitored Cryoballoon Ablation

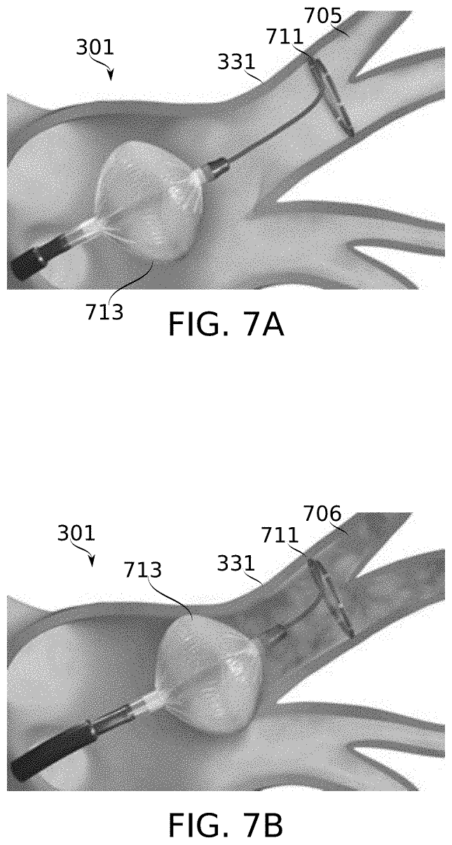

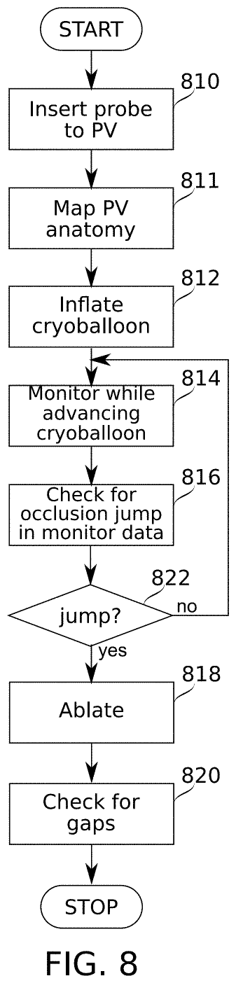

Reference is now made to FIGS. 7A-7B, which schematically represent stages in cryoablation including insertion of a lasso catheter probe 711 into a pulmonary vein 331 of a left atrium 301, and conversion of blood flow 705 into blocked flow 706 as a cryoballoon 713 is pressed firmly up against the ostium leading into pulmonary vein 331, according to some embodiments of the present disclosure. Reference is also made to FIG. 8, which is a schematic flowchart describing a method for electrical monitoring of the flow blockage 706 shown in FIGS. 7A-7B, according to some embodiments of the present disclosure.

At block 810, in some embodiments, the electrode lasso of a catheter probe configured like the lasso-and-balloon probe 711 of FIG. 7A is inserted to a pulmonary vein (PV). At block 811, the PV anatomy is mapped, for example to verify that the geometry is of an appropriate size and shape to allow use of the cryoballoon 713 to make a complete ablation around the ostium of the PV.

At block 812, in some embodiments, the cryoballoon is optionally inflated, and the catheter probe 711 positioned in a state like that shown in FIG. 7A—balloon inflated, but not yet positioned to press against the PV ostium. Alternatively, in some embodiments, the balloon is advanced into position while remaining deflated, and is gradually inflated in place.

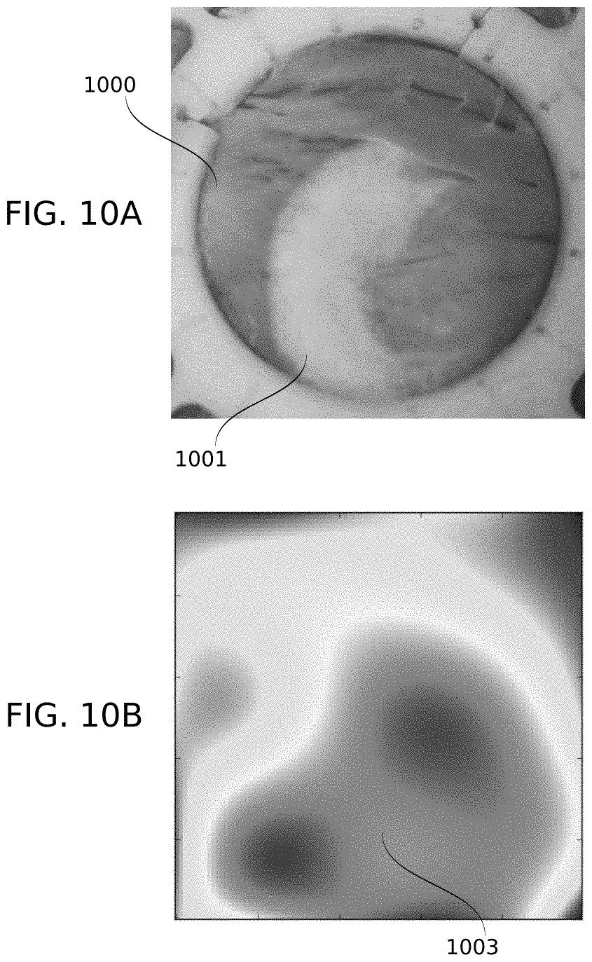

At block 814, the cryoballoon is advanced towards (and/or inflated within) the PV ostium while electrically monitoring voltages generate from electrodes of the lasso catheter probe 711 using those same electrodes. During advancing/inflating, at block 816, a check is made for an occlusion jump in the monitoring data. An occlusion block, in some embodiments, comprises a relatively large and sudden change in voltage, characteristic of the moment when the vein becomes occluded. In some embodiments, the jump comprises a change within about 100-250 msec of at least 3× the sampling noise. This potentially corresponds to a moment when a substantially full seal is formed (e.g., a seal covering at least 99% of the cross-sectional area of the occluded lumen, and/or preventing at least 99% of flow). In some embodiments, this rapid change is preceded by a somewhat slower change of about the same amplitude, occurring over the course of about 500-1500 msec. In some embodiments, the rapid change is followed by an overshoot and partial reversal of the change, over the course of about 300-600 msec. Such occlusion jump behavior has been observed by the inventors in association with the completion of sealing of the PV ostium by the advancing cryoballoon. At block 822, if the jump has not yet been noted, the flowchart returns to block 814. Otherwise, the flowchart continues at block 818 with cryoablation (e.g., filling of the cryoballoon with cryogenic fluid to induce a preferably circular lesion around a periphery of the PV ostium). Optionally, after the completion of ablation, electrodes of the lasso probe (or another probe) are used (at block 820) to check the resulting lesion for gaps, for example using impedance measurements. An example of data resulting from such a check in a phantom pig heart is provided in FIGS. 10A-10B. Optionally (not shown) gaps are repaired by additional cryoballoon lesioning and/or targeted RF lesioning.

A potential advantage of the method of FIG. 8 for monitoring occlusion is to avoid a need for X-ray imaging and/or contrast medium injection to verify that a good balloon-tissue contact has been accomplished.

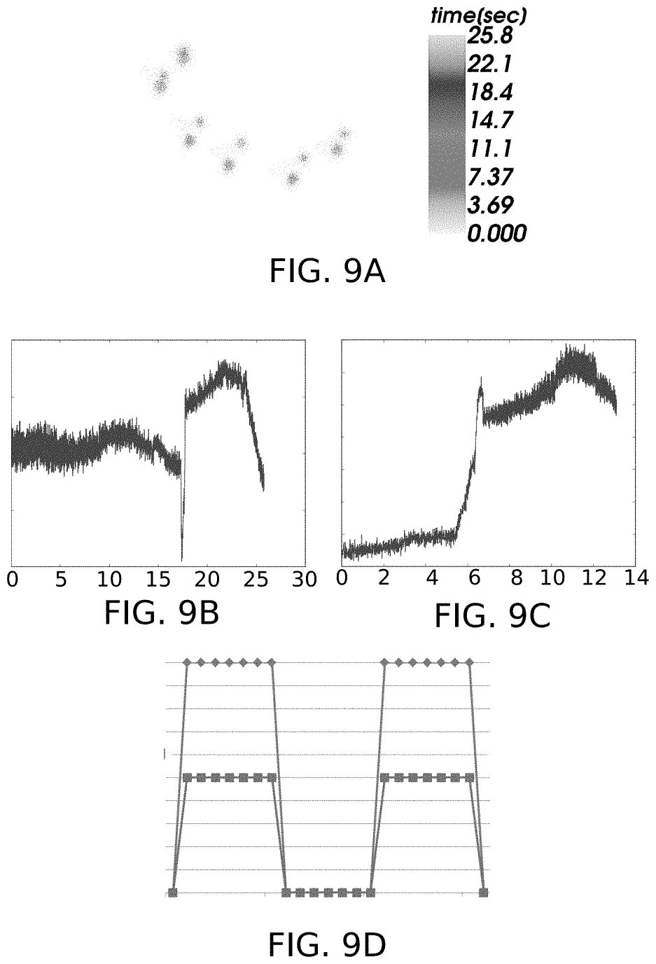

Reference is now made to FIGS. 9A-9D, which schematically represent test results of the method of FIG. 8, according to some embodiments of the present disclosure.

FIG. 9A shows changes in sensed voltage at a two particular frequencies (of an optional multiplicity of frequencies which may be used), from a plurality of electrodes such as the lasso electrodes of catheter probe 711. Results from six electrodes are shown. Earlier-recorded values from each electrode are more yellow/reddish; later-recorded values more bluish. It may be observed that there appears to be a relatively sudden jump at around 18 seconds from a cluster of reddish (early) voltage values to a cluster of bluish (later-recorded) voltage values. These jumps correlate with the moment of sealing contact between the cryoballoon (a Medtronic Arctic Front cryoballoon) and a water-immersed pig heart phantom, as indicated by reduction of fluid flow maintained by a syringe attached to an open tube through the phantom PV to zero.

FIGS. 9B-9C show onset and offset of the voltage jump for a single electrode, including a voltage jump in FIG. 9B when flow was stopped by the cryoballoon (at about 18 seconds), and other jump at about 7 seconds in FIG. 9C when flow was resumed (by deflation/moving of the cryoballoon).

FIG. 9D shows second-by-second correlations between measured flow velocity (square data points) and the voltage jump signal (diamond data points), strengthening the case for a causal association between balloon sealing and the voltage jump.

Reference is now made to FIGS. 10A-10B, which respectively represent visual results of cryoablation in vitro on a muscle tissue preparation 1000 (FIG. 10A), and dielectric assessment of the same results (FIG. 10B) which reveals a potential gap 1003 in the apparently well-ablated region 1001.

Another potential advantage of the method of FIG. 8 is that the electrodes of the lasso are nearly in position to be repositioned to measure the possible presence of ablation gaps, so that remedial action can be taken immediately, potentially before the full onset of tissue reactions such as edema which can interfere with the effectiveness of subsequent ablation attempts.

The light-colored region 1001 of FIGS. 1A-1B is discolored due to previous exposure to cryoablation (the lesion is not circular because of the flat geometry of the test preparation). However, upon dielectric measurement of tissue properties in the area, it was found that a partial gap indicated in region 1003 remained. Dielectric measurement of tissue lesion properties is described, for example, in International Patent Publication No. WO2016/181318, entitled LESION ASSESSMENT BY DIELECTRIC PROPERTY ANALYSIS, and published on Nov. 17, 2016.

Single Catheter Transseptal Access and Left Atrium Ablation

Reference is now made to FIG. 11, which is a schematic flowchart describing a method for single-electrode transseptal penetration from the right to the left atria, followed by ablation within the left atrium, according to some embodiments of the present disclosure.

Blocks 1110-1114, in some embodiments, correspond to blocks 510, 512, and 514 of FIG. 5.

At block 1110, in some embodiments, a catheter probe 11 comprising at least a tip electrode configured act as an RF ablation probe is navigated to an IAS by any suitable method, for example as described in relation to FIG. 3A, herein. At block 1112, the probe is moved over the IAS while making dielectric measurements, and at block 1114, the fossa ovalis (or patent foramen ovale, according to the anatomy) is identified from the dielectric measurements. At block 1116, the probe is moved to the fossa/PFO (if it is not there already). At block 1122, a determination is made as to whether the ablation catheter probe 11 can already cross the septum (e.g., because there is a PFO). If not, then at block 1118, the RF ablation electrode of the catheter is activated to ablate at the fossa ovale. Optionally, ablation settings used are similar to those used in normal transmural ablation for AF treatment. Optionally, the ablation settings are more aggressive, however, in order to achieve substantial mechanical weakening of the IAS structure which is normally preferably avoided in AF lesion treatments. Potentially, the ablation weakens the already thin fossa ovalis sufficiently to allow the catheter probe 11 to penetrate it through the use of blunt force. From which ever branch of the method, at block 1120, in some embodiments, the catheter probe is pushed across the IAS. The catheter is navigated into position to perform ablation treatments, and at block 1124, in some embodiments, ablation in the left atrium (e.g., ablation to encircle PVs with ablation lines) is performed.

Potentially, crossing the IAS without a transseptal needle is advantageous economically, e.g., for requiring fewer tools and/or fewer tool changes during a procedure. Crossing by applying RF energy is optionally performed, for example, with a dedicated Baylis system and/or a standard RF generator.

Whole Treatment Procedures without X-Ray and/or Other Imaging Assistance

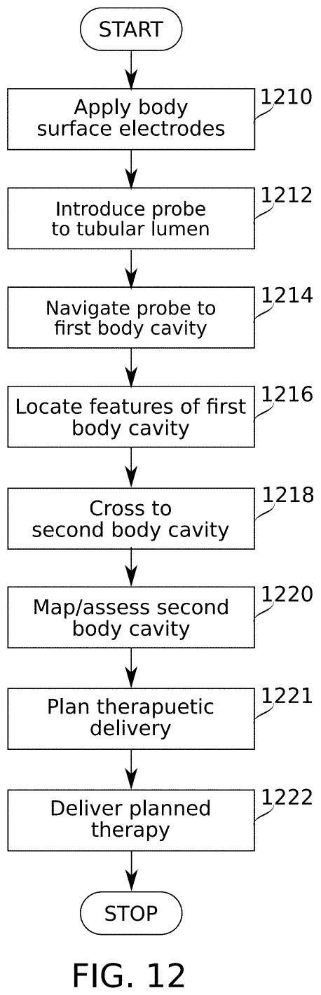

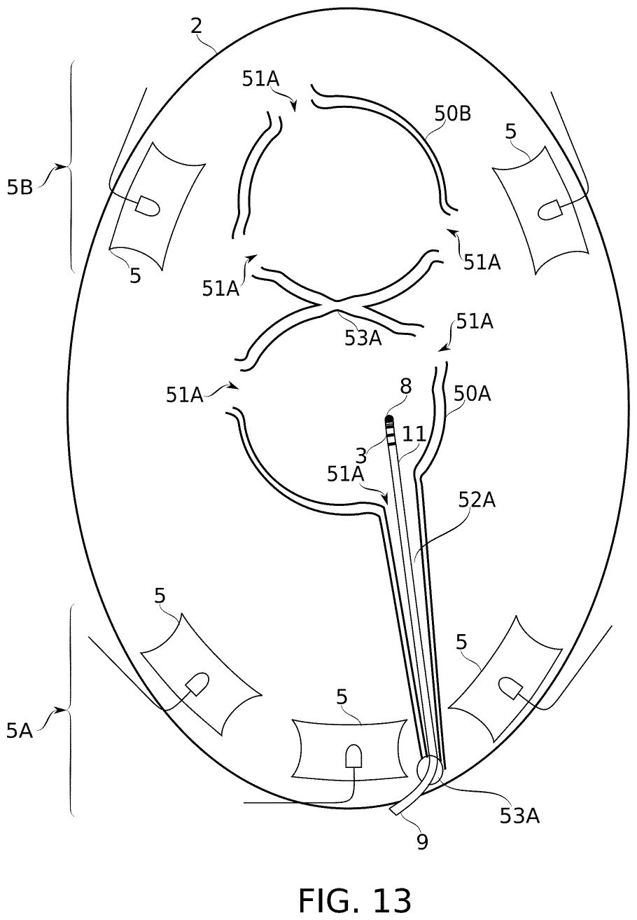

Reference is now made to FIG. 12, which is a flowchart describing a method of using an electrode probe 11 to navigate to and treat a target in a body tissue cavity (e.g., tissue 50B), according to some embodiments of the present disclosure. Reference is also made to FIG. 13, which schematically illustrates components and body structure elements described in relation to the method of FIG. 12, according to some embodiments of the present disclosure.

The method of FIG. 12 relates to the sequential visitation by electrode probe 11 of two body cavities (as well as its initial traversal to those body cavities), in illustration of different types of tasks which may be performed using one or more of the types of electrical field-based navigation and/or reconstruction methods outlined, e.g., in relation to block 1214. Optionally one or more auxiliary probes (not shown) are used together with the electrode probe 11 to perform assisting functions. The first body tissue cavity may be understood as a transit region, which is entered by an electrode probe, and from which a probe exit direction is to be identified, selected, and safely passed along. The first body tissue cavity may also be involved in the performance of preparatory steps, for example, the positioning of additional probes for assisting in a procedure. The second cavity may be understood as a treatment region, in which one or more clinical tasks such as diagnostic measurement, ablation treatment or another clinical task is to be performed. It should be understood that there may also be treatment tasks performed in the first body tissue cavity, and the second cavity is optionally itself a transit region to a further body lumen (e.g., body tissue cavity or tubular lumen). It should be understood, however, that treatment tasks are optionally performed in a single body tissue cavity, e.g., the first body tissue cavity.

Also described is the passage of one or more tubular lumens which must be navigated to reach the first and/or the second body tissue cavity. While the method of FIG. 12 relates to first and second body tissue cavities, this is for the sake of illustrating a variety of actions which may be performed during a procedure under the guidance of electrical fields sensed by electrode probe 11. It should be understood that there is no particular limitation to visiting two different body tissue cavities; there may be one, two or more such cavities (nor is solid tissue excluded, for example as further described hereinbelow).

It should be noted in particular that the method of FIG. 12 (or a variant thereof) is performed without the use of imaging by an externally placed imaging sensor, and in particular without the use of X-ray (or any other ionizing radiation) or US imaging. In some embodiments, the method is performed without use of injected contrast agent.

In some embodiments, the method of, for example FIG. 12 (or a variant thereof) allows performing a method of allocating operation rooms to operation procedure comprising selecting an X-ray unshielded room (optionally one that does not contain an X-ray machine) and allocating the selected room to a catheterization procedure (optionally while the room remains without an X-ray machine), thereby freeing X-ray shielded rooms to operation procedures for which X-ray shielding is essential; for example, essential under applicable safety regulations.

In some embodiments, a room including a catheterization system used for performing, for example, the method of FIG. 12 (or a variant thereof) includes a processor, wherein the processor is connected to: a display, an input for receiving from an intra-body electrode probe measurements of electrical fields, and a data analyzer connected to the input and configured to generate an image from the measurements. In some embodiments, the room includes a support for a patient with which the catheterization system is used. For example, in some embodiments, the catheterization system is configured to guide a catheter inside a patient supported by the support, and arranged to be operable by a physician when the physician is viewing the display. The walls of the room are X-ray penetrable, and the room may include at least one X-ray penetrable window.

In some embodiments a system is provided for use in performing the method of FIG. 12. In some embodiments, the system comprises a radiation source (e.g., an electrical field generator/measurer 10), configured to generate non-ionizing electromagnetic radiation; a catheter (for example, a catheter 9). In some embodiments, the system comprises an electrode probe configured to apply non-ionizing electromagnetic radiation generated by the radiation source to a penetrated blood vessel of a patient. In some embodiments, the system comprises a data analyzer, configured to generate guidance for movement of the catheter from a vascular obstruction or branch encountered by the electrode probe after an insertion of the probe into the patient to a target beyond the vascular obstruction or branch and along a planned catheter route. Optionally, the guidance is generated based on measurements indicative of interactions of tissue near the electrode probe with the non-ionizing electromagnetic radiation applied by the electrode probe. Optionally, the data analyzer comprises estimator services 22 of FIG. 2A, optionally with estimator result 212 being used as the guidance. Optionally, the guidance is displayed using a user interface 40. In some embodiments, the system comprises a catheterization system configured to guide the electrode probe inside a patient, and arranged to be operated by a user when the user is receiving the guidance generated by the data analyzer. Optionally, the system comprises a support for supporting a patient (for example, a patient bed).

In some embodiments, only electrical field sensing by an intrabody electrode probe is used to guide navigation of the probe and body lumen reconstruction, from the time the electrode probe is introduced into the body, to the time that clinical tasks are performed in a target region (e.g., the second body cavity). This includes, in some embodiments, guidance and body lumen reconstruction without the use of a reference diagnostic image, such as an image of anatomy obtained prior to a procedure by CT or MRI. Optionally, e.g., for purposes of display presentation, position data obtained using electrical field sensing (and/or a 3-D reconstruction of anatomy derived therefrom) are/is of sufficient detail that they can be matched, without additional imaging, to a general (e.g., atlas-derived) body anatomy. The matching is optionally based, for example, on general homologies (e.g., extents of tubular lumens, and/or general relationship of body chambers to one another).

While embodiments of FIG. 12 are described with particular reference to catheter-borne electrode probes, and body tissue cavities tubular lumens, and/or walls separating cavities, it is to be understood that navigation and/or mapping is optionally performed by other types of instruments, in other tissues which those instruments are capable of navigating/mapping and/or treating. For example, the electrode probe may comprise a portion of a cutting tool such as a needle, scalpel, or laser probe which is configured to cut a way through solid tissue. Optionally, the electrode probe is provided as an add-on to such a tool (e.g., as a sticker, pull-over add-on, or another configuration).

With respect to the descriptions of FIG. 12, a body tissue cavity 50A, 50B comprises a widened lumenal region and its immediately surrounding body tissue (e.g., a heart atrium, heart ventricle, bladder, or renal pelvis), connected into by one or more smaller-diameter tubular lumens 52A (e.g., blood vessel(s), ureter, urethra, or other tubular tissue structure) at body tissue cavity apertures 51A.

The flowchart of FIG. 12 begins, and at block 1210, in some embodiments, one or more body surface electrodes 5 are placed on the body surface of a patient body 2 in preparation for introduction of the electrode probe 11 to a blood vessel. The body surface electrodes 5 are optionally placed in one or more electrode sets 5A, 5B which are configured to transmit electrical fields through body 2, for use in guiding the navigation of the electrode probe in one or more body tissue regions. Use of the transmitted electrical fields for mapping and/or navigation is optionally in one or more of several modes, described further, for example, in relation to block 1214. In some embodiments, one or more electrodes may be provided on an additional intrabody probe (i.e., additional to probe 11) for transmitting electrical fields in body 2. The additional intra-body probe may be provided in an additional body cavity not subject to treatment; for example: when treating the heart, additional intra-body probe may be provided in the coronary sinus.

In some embodiments, for example, introduction of electrode probe 11 to the interior of patient body 2 is via an incision 53A into a femoral vein or artery (optionally corresponding to tubular lumen 52A). Optionally, a set of one or more body surface electrodes 5 is placed around the region of the hip (or another region near incision 53A), and configured to transmit electrical fields through the region of the hip which may be used as a basis for mapping by and/or navigation of the electrode probe 11 at an initial stage of the procedure.

In some embodiments, body tissue cavity 50B targeted for treatment comprises a chamber of the heart. Optionally, a set 5B of one or more body surface electrodes 5 is placed and configured to transmit electrical fields through the region of the heart which may be used as a basis for reconstructing by, mapping by, and/or navigation of the electrode probe during one or more later stages of the procedure.

Other body tissue targets for the procedure of FIG. 12 may include, for example, a kidney via the urethra, bladder, and a ureter. Set(s) of body surface electrodes 5 may be placed and configured accordingly to the requirements of the particular procedure and body tissue targets.

At block 1212, in some embodiments, the electrode probe 11 is introduced (e.g., via a short introducer) to a first tubular lumen 512A (e.g., a blood vessel). The blood vessel may be, for example, a femoral vein, femoral artery, radial artery, or another blood vessel. While FIG. 12 is described primarily with reference to navigation of an electrode probe 11 via blood vessels, in some embodiments (e.g., for navigation to a kidney), introduction is via another body lumen, such as a urethra.

The electrode probe 11, in some embodiments, is an electrode probe 11 such as is used with (e.gl, advanced at the distal end of) a catheter 9 in in catheter procedures; and may be, for example, a guide wire provided with electrodes (for example, electrode-equipped guidewire 1100 of FIG. 17A), or an electrode probe of an electrophysiology catheter.

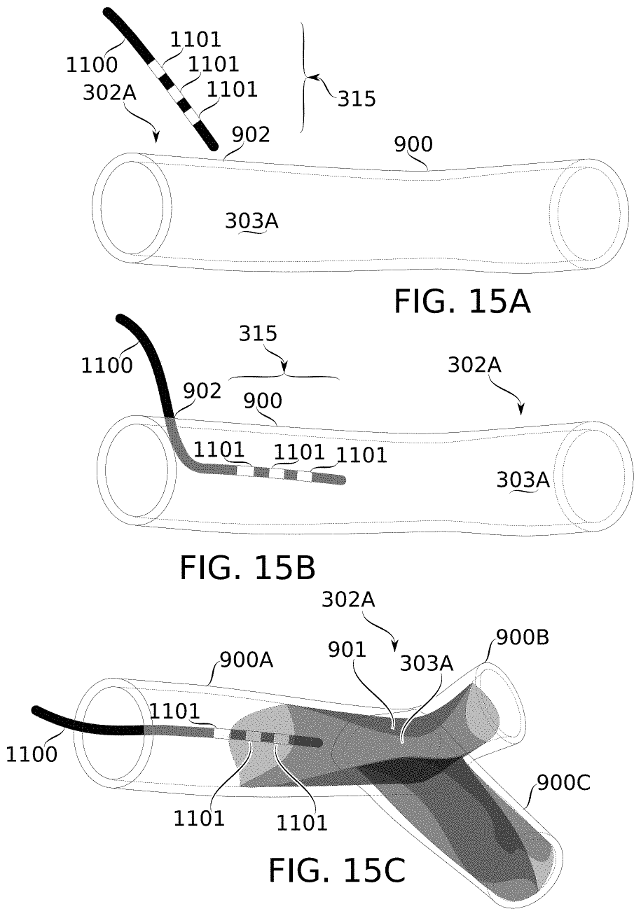

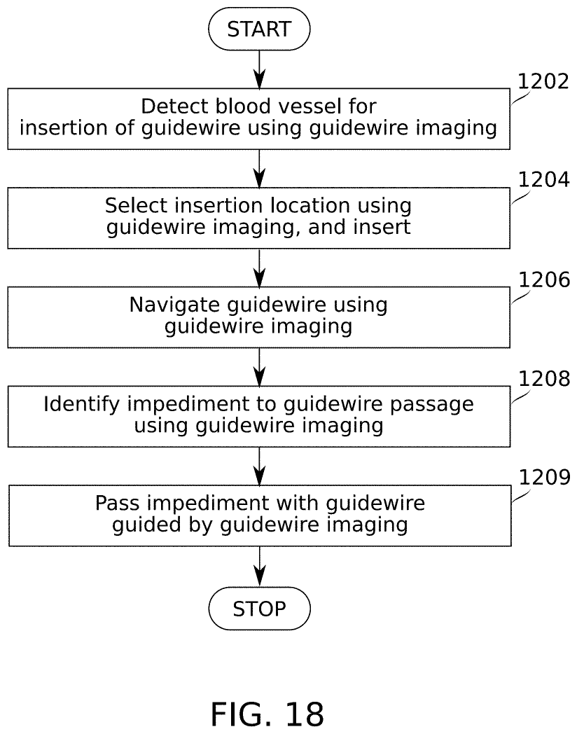

In some embodiments, introduction of the electrode probe 11 is performed with use of electrical field sensing by the electrode probe 11 to provide guidance as to the position of insertion, for example, to produce an image used as guidance. This is described for an electrode-equipped guidewire 1100, for example, in relation to FIGS. 15A and 18.

At block 1214, in some embodiments, the electrode probe 11 is navigated via one or more tubular lumens 52A (e.g., blood vessels or other body lumen(s) as appropriate) to a first body tissue cavity 50A. In some embodiments, this includes navigation of an electrode-equipped guidewire 1100 through vasculature and potentially through vascular obstructions while imaging (and optionally providing the imaging for guidance of electrode probe 11), for example as described in relation to FIGS. 15B-15C, 16 and 18. A vascular obstruction is any abnormal structure that obstructs blood flow in a blood vessel, for example, plaque, vascular stenosis, or a growth.

Optionally, any one or more of several modes of electrical field-guided navigation and/or reconstruction are used in providing guidance used in the operations of block 1214, 1216. For example:

Time-varying electrical fields transmitted between body surface electrodes 5 through body 2 are treated as being approximately linear in some regions, thereby establishing (in those regions) a basis for coordinate axis-like measurements. As an electrode moves along a voltage gradient of an electrical field, it measures different voltages (optionally, voltages which provide measurements of impedance). Voltage measurement may thus be used as a measure of position within the electrical field, and thus within a body that the electrical field is transmitted through. With a plurality of such electrical fields crossing (between different subsets of body surface electrodes 5) at different angles, a spatial coordinate system may be established. Optionally, corrections for non-linear features of the electrical fields are applied to provide a spatial calibration. Electrical fields are distinguished, for example, by oscillating at different frequencies, and/or by time multiplexing.

Where the linear approximation of the spatial arrangement of time-varying electrical fields is not suitable (e.g., due to local inhomogeneities in dielectric properties, and/or due to proximity to a non-linear electrical field region, such as a region near a transmitting electrode), measurements of crossing electrical fields may still be treated as labeling particular regions where they are made, and as identifying movements. Particularly with respect to navigation using non-linear arrangements of electrical fields, those electrical fields are optionally transmitted from electrodes other than body surface electrodes 5. For example, an electrode probe may be placed in a coronary sinus 312 or other body lumen, for example as described in U.S. Provisional Patent Application No. 62/449,055 filed Jan. 22, 2017 entitled “CORONARY SINUS-BASED ELECTROMAGNETIC MAPPING”, the contents of which are incorporated by reference herein in their entirety. Optionally, the electrical probe being navigated is also used to transmit electrical fields used for the navigation, with changes in measurements of the self-transmitted electrical fields in different body positions being used as characteristic of those different body positions.

Movements through an electrical field-labeled space (however the electrical fields are generated) are optionally calibrated to spatial position using knowledge of the spacing of electrodes 3 on the electrode probe 11 itself. This spacing acts as a “self-ruler” that helps to provide a constraint on how far apart two nearby electrical field measurements actually are. This constraint in turn helps in the reconstruction of larger distances (and optionally of whole spaces) as an electrical probe 11 moves through a larger range of positions; for example, as a more proximal electrode moves to occupy a position which a more distal electrode just left. In addition to the distance (and optionally spatial position in a plurality of directions) constraint, the reconstruction of body cavity may be further assisted by relying on one or more additional constraints, for example, by assuming that the electrical fields are locally coherent (e.g., that they vary in such a way that closer positions are also closer in their measured electrical field properties; and optionally also that this holds true for electrical field gradients as well as the direct measurements themselves). The constraints may be weighted and/or combined, so that neither absolutely dominates the other (allowing, e.g., compensation for measurement error). Methods for performing navigation and reconstruction in this manner are described, for example, in U.S. Provisional Patent Application No. 62/445,433 filed Jan. 12, 2017 entitled “SYSTEMS AND METHODS FOR RECONSTRUCTION OF INTRA-BODY ELECTRICAL READINGS TO ANATOMICAL STRUCTURE”, the contents of which are incorporated herein by reference in their entirety. This type of reconstruction and/or navigation are of particular use in reconstructing the shapes of body cavities from electrical field measurements, for example as described in relation to blocks 1216, 1218, and 1220.

Another method of reconstructing a body tissue cavity (e.g., 50A, 50B) comprises measurement of the spatial distribution of electrical field gradients in a relatively restricted volume of the body tissue cavity 50A, 50B. From this spatial distribution, optionally together with scaling information (such as the distance it takes to cross the cavity in one direction), remote wall positions of the body tissue cavity 50A, 50B may be reconstructed. Optionally, the remote wall positions are extrapolated from measurements in the relatively restricted volume based on observations that electrical field lines tend to be close together (current is denser) in directions that “point at” wall apertures and other more distant wall features, compared to directions that “point at” closer wall features. In some embodiments, reconstruction is performed using a variant of the “inverse method”; e.g., seeking by iteration of a cavity wall model and reduction of error to find a tissue configuration that is consistent with the locally observed electrical field gradient distribution. Either type of reconstruction is also referred to herein as “remote electrical field imaging”. This type of reconstruction is described, for example, in U.S. Provisional Patent Application No. 62/546,775 filed Aug. 17, 2017 entitled “FIELD GRADIENT-BASED REMOTE IMAGING” the contents of which are incorporated herein by reference in their entirety.

Position may also be determined with respect to variations of electrical field properties (for electrical fields transmitted from body surface electrodes 5, from internally placed electrodes of another electrode probe, and/or from the electrode probe 11 itself) as measured at certain landmarks and/or waypoints. Additionally or alternatively, contact with body tissue may be identified electrically, for example, by noting differences in impedance measurements at the time of contact and/or approach to contact. These measurements are optionally performed using transmission of electrical fields from one or more of the electrodes 3 on the electrode probe 11 itself. For example, at vascular branch points, and/or at widenings where a blood vessel enters a heart chamber, there may be a characteristic change in impedance noted due, e.g., to how free electrical current is to flow through the surrounding volume of blood. Other characteristic changes in impedance may occur as a blood vessel passes nearby another tissue type (such as bone, esophagus, and/or lung) which affects the local dielectric environment. Another characteristic change may occur, e.g., due to characteristic curvatures of the tubular lumen, for example, in the aortic arch. Characteristic feature navigation is described, for example, in International Patent Application No. PCT IB2017/054263 filed on Jul. 14, 2017, and entitled “CHARACTERISTIC TRACK CATHETER NAVIGATION”, the contents of which are incorporated herein by reference in their entirety.

Any combination of the above-mentioned and described methods of electrical field-guided navigation, imaging, and/or reconstruction may be performed. For example, reconstruction is not necessarily performed in regions where the main reason for visiting with electrode probe 11 is to transit the region on the way to somewhere else. E.g., in some embodiments of the operations of block 1214, it may be sufficient to indicate orientation of the electrodes 1101 (an orientation under control by steering of guidewire 1100) to a direction which is relatively free of impediment, thereby indicating that a current orientation of guidewire 1100 is suitable for being advanced further. This may nevertheless constitute imaging, e.g., insofar as an image may be created of measurements as a function of catheter orientation and/or position. Where reconstruction is performed (for example, in order to more fully characterize a blood vessel obstruction, and/or find targets for transseptal crossing and/or treatment), it may be performed roughly for regions of a body tissue cavity of less direct interest, and in a more detailed fashion for regions where more precise knowledge of anatomical structure is needed. It is noted again that any of these methods may be performed without the use of an externally supplied image reference, such as a CT, MRI, X-ray, or ultrasound (e.g., IVUS) image.

With particular reference to the navigation of block 1214, characteristic feature navigation may be of particular use as guidance in moving an electrode probe through tubular lumen(s) 52A. Through the extent of a tubular lumen, precise positioning optionally is not critical, so long as certain waypoints (e.g., valves, vascular junctions, entrances into a heart chamber) can be identified when reached.

At block 1216, in some embodiments, the first body tissue cavity 50A is explored using the electrode probe 11, in sufficient resolution to locate (via an image produced during the exploration, and used as guidance) an exit leading toward a second body tissue cavity 50B. Optionally, any of the electrical field-based navigation and/or mapping methods mentioned in relation to block 1214 may be used.

In some embodiments, identifying the exit from the first body cavity includes a phase of anatomical orientation within the first body cavity, comprising the identification of one or more landmarks that help indicate the general position of the exit. Pinpointing and transiting the exit, in some embodiments, may include further operations. Potentially there is risk of damage to vulnerable structures during any of these operations.

Thus, features may be mapped for one or more guiding uses; for example:
Landmark features may be mapped to help orient the position of the electrode probe 11, and/or the position of other structures relevant to movements of the electrode probe 11.
There may be preparation work to perform, such as positioning of auxiliary catheters, before treatment in the second body tissue cavity 50B can be performed.
Features (exit targets) targeted for receiving exit-directed movements of the electrode probe may be located, characterized and/or acted upon.
Features (structures and/or regions) may be located which are vulnerable to damage and/or associated with adverse incidents if disturbed.

Particular features which are mapped depend on the anatomical type of the first body tissue cavity 50A. In some embodiments, for example, the first body tissue cavity 50A is a right atrium 303 (shown, for example, in FIGS. 4A-4C), and a subsequent goal for the navigation of electrode probe 11 is to transit electrical probe 11 to the left atrium 301 (as second body tissue cavity 50B), via the inter-atrial septum 310.

The right atrium 303 has a characteristic arrangement of features such as vascular and valvular apertures (e.g., the superior and inferior vena cava 320, 316; coronary sinus 312; and tricuspid valve). The positions of these features may help to locate the position of the fossa ovalis 311 or patent foramen ovale (depending on which is present) which is to be targeted in crossing the inter-atrial septum 310. Locating of the crossing point and crossing of the inter-atrial septum 310 is described, for example, in relation to FIGS. 4A-6 and 11. It is a potential advantage to be able to cross the inter-atrial septum 310 without reliance on external imaging, e.g., on ultrasound monitoring.

There may also be a concern during a procedure to avoid structures vulnerable to damage and/or at risk to cause complications if disturbed. In crossing the after-atrial septum, 310, for example, care is preferably taken to avoid interacting with the aorta lying behind it. Puncture of the aorta, for example, is a serious bleeding complication. In some embodiments, the location of the aorta can be determined (e.g., using remote electrical field imaging) by noting positions where electrical field gradients are distorted, due to the large mass of blood and/or vascular wall tissue just on the other side of the heart wall.

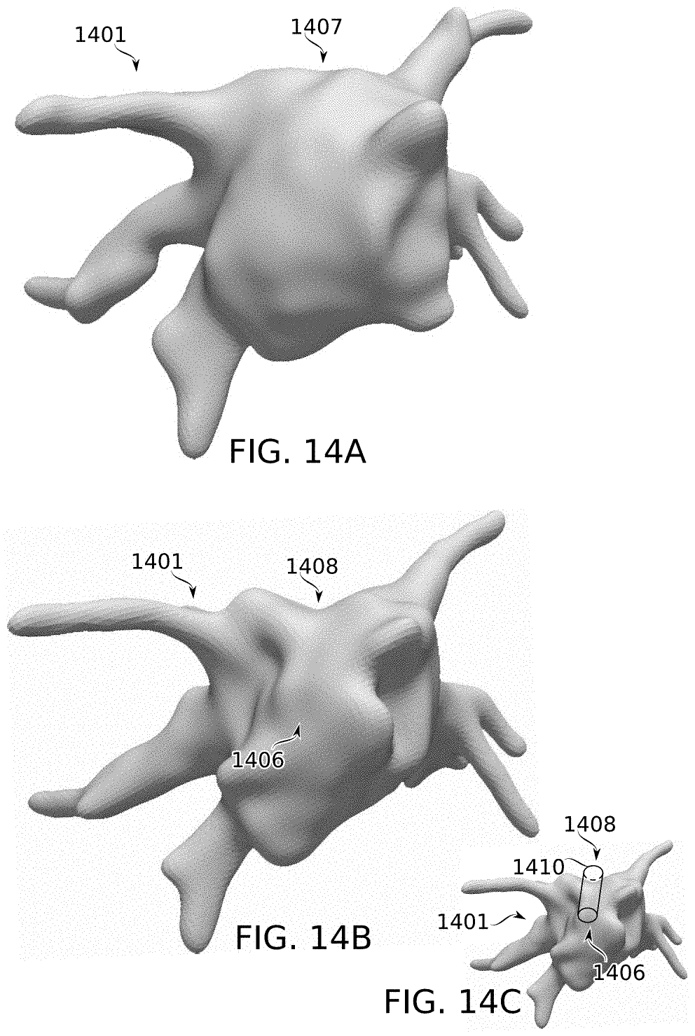

Brief reference is now made to FIGS. 14A-14C, which illustrate a result of electrical mapping of a phantom left atrium 1401 (a plastic resin model immersed in a water-filled tank), with and without a phantom aorta 1410 (saline-filled syringe) located alongside, according to some embodiments of the present disclosure. At region 1407 in FIG. 14A, the mapped shape of the wall is substantially isomorphic to the actual shape of the phantom wall. However, in FIG. 14B, the same region is shown with an indentation in a region between wall region 1408 and wall region 1406. This indentation may correspond to the position of the phantom aorta 1410, as indicated in FIG. 14C.

The indentation, as results in the case of FIG. 14B, may be understood as a consequence of the use of the “self-ruler” method of mapping described above; and for example, in U.S. Provisional Patent Application No. 62/445,433 filed Jan. 12, 2017 entitled “SYSTEMS AND METHODS FOR RECONSTRUCTION OF INTRA-BODY ELECTRICAL READINGS TO ANATOMICAL STRUCTURE”. In this method, the self-ruler constraint (that is, known fixed distances between a plurality of sensing electrodes 3) and the coherence constraint may be weighed against each other in a common error function.

For purposes of explanation, but without commitment to a particular theory, electrical field distortion is caused in proximity to the phantom aorta. The distortion is due, for example, to local dialectical property differences; in the case of the phantom, saline has different (e.g., about 2× more conductive) conductivities than the water around it. In the case of the aorta, the aorta comprises a blood and tissue concentration adjoins the cardiac wall which is different in composition than other tissue regions adjoining the cardiac wall.

The effect of the distortion on reconstruction depends on the reconstruction method. In one example, it may be understood as straining the coherence constraint used in reconstruction, e.g., by producing unexpectedly sharp changes in voltage gradient that look like “incoherence”. There is thereby produced an error indication during reconstruction, even though the actual measurements are correct for their positions. Since the coherence constraint and the “self-ruler” constraint may be tied into a joint error function through their relative weighting, the reconstruction algorithm compensates for the error indication by allowing coherence error to distribute through both constraints. Effectively, the “self-ruler” may be allowed to change in size by a small amount to reduce coherence error. This results in the cavity wall, when it is encountered by the moving electrode probe 11, being placed in the reconstruction at a slightly offset position, resulting in a local distortion from its true shape. In the case of the phantom aorta, the distortion takes the form of an intruding section of a cylindrical surface. Insofar as it is separately known that such a distortion is not consistent with how chamber walls are actually formed, the distortion reveals the position of the aorta. It should be understood that this is just one method of visualizing aorta position, demonstrating that effects of an adjacent aorta can be readily detected. The underlying distortion in electrical fields is optionally extracted by another method, e.g., by noting regions in the mapped space where the “self-ruler” constraint is systematically distorted in a manner consistent with a nearby aorta.

Another example of a reconstruction method showing effects due to “beyond the wall” structural differences uses the remote electrical field imaging method. Here, the bending of the fields by a structure (e.g., the phantom aorta) affects the image produced, insofar as adjustments in field gradient are attributed by the reconstruction process to how near or far a body tissue cavity wall is.

Returning now to the discussion of block 1216: in some embodiments, electrical mapping and/or navigation in a later phase of the procedure is to be performed with the use of electrical fields transmitted from electrodes placed in the coronary sinus 312 on a catheter-borne auxiliary electrode probe. Optionally, mapping is performed to locate the coronary sinus 312 ostium and/or the Thebesian valve, and then the auxiliary electrode probe is navigated into the coronary sinus 312. The auxiliary electrode probe is optionally also the probe used to identify the coronary sinus 312 ostium.

In some embodiments, the crossing is from the other direction. For example, an electrode probe, in some embodiments, is navigated from an arterial direction into the left ventricle and atrium, and then across the inter-atrial septum 310 to the right atrium 303. In this case, there is a task of crossing the aortic valve. In some embodiments, determination of a position and/or timing for crossing the aortic valve is optionally performed using electrical field measurements. For example, electrical properties in the region of the valve may vary cyclically as the valve opens and closes, and may vary more in regions which the valve pulls radially away from the most. Moreover, it is a potential advantage to cross at a radial position at which the opening is clearest of obstructions (e.g., calcifications), to avoid possibly damaging the valve and/or dislodging material into the bloodstream.

In some embodiments, the urinary tract is to be navigated, e.g., for the treatment of a kidney stone. In this case, the first body tissue cavity comprises the bladder, entered from the urethra, and exited via the ureter toward the renal pelvis of the kidney.

At block 1218, in some embodiments, the electrode probe is crossed from the first body tissue cavity 50A to the second cavity 50B. As illustrated in FIG. 13, the crossing is at the wall weak point 53A (e.g., as in for crossing between the right and left atria, or in the reverse direction). Locating and crossing of the inter-atrial septal wall is described, for example, in relation to FIGS. 4A-6 and 11. Optionally, the crossing is along a tubular lumen, for example as in the crossing from a bladder to the renal pelvis.

At block 1220, in some embodiments, the second cavity 50B is mapped using an electrical field mapping technique, and optionally further assessed in preparation for treatment. Mapping can comprise any of the electrical mapping methods described in relation to block 1214; for example, the “self-ruler” method, the method of mapping more distant wall positions based on local distortions in electrical field gradient, the method of using crossed electrical fields as approximate linear axes, or any suitable combination thereof.

If crossing from the right atrium into the left atrium, the details mapped may include, for example, the pulmonary veins 302, mitral valve 308, left atrial appendage 319, and warfarin ridge 306 (also called the left atrial appendage ridge). It is noted that the method of mapping (or “remote electrical field imaging”) more distant wall positions based on local distortions in electrical field gradient in particular is well-suited to this phase of exploration, since it can discover positions of structures without requiring taking the time to make a visit in detail to each. As another potential advantage, the left atrial appendage potentially hosts thrombotic material which could be dislodged into the bloodstream by contact from electrode probe 11. Serious complications such as stroke may result. Reducing exploratory movements of the electrode probe 11 potentially also reduces a risk of this type of incident. Also, remote electrical field imaging potentially allows establishing the position of the plane of the mitral valve, so that it can be avoided to reduce a risk of valve damage during the procedure.

Optionally, once a basic map of the second body chamber 50B has been established, the map may be refined in detail by closer visiting of targets of particular interest. Ablation around the pulmonary veins 302 to treat atrial fibrillation provides an example of a treatment which may be carried out in the left atrium 301. The pulmonary veins may be visited using electrode probe 11 and electrically mapped in order to more determine more particularly and/or verify their topography. The topography can have an effect on how later treatment is carried out. In particular, the shape of the warfarin ridge, adjacent to the PVs can affect the design of an ablation line which is intended to electrically isolate them from surrounding tissue.

Optionally, further assessments are carried out in preparation for the performing of a treatment in the second body cavity 50B. A task in the left atrium which may be performed as part of assessment of the second body cavity 50B is to measure baseline flow of blood through the PV. For example, electrical monitoring of flow blockage using electrical field measurements is described herein in relation to FIG. 8. Electrical monitoring of flow may allow avoiding flow assessment by dye injection and angiographic imaging.

Another task which may be performed as part of assessment is to assess the state of tissue in the region of ablation. For example, the pattern of electrical transmission activity can be assessed. Other aspects of tissue state can be assessed, for example, using impedance measurements to determine tissue health, tissue thickness (e.g., thickness of a cardiac muscle wall), or another property, for example as described in U.S. patent application Ser. No. 15/573,493 filed May 1, 2017 and entitled “LESION ASSESSMENT BY DIELECTRIC PROPERTY ANALYSIS”, the contents of which are included herein by reference in their entirety.

Safety checks may also be performed, for example, to locate the aorta's position with respect to the left atrium (substantially as already described for the right atrium), and/or to locate the current position of the esophagus, which, as “a tube of air”, also may have distorting effects on local electrical field gradients. The esophagus can be damaged by treatments such as RF ablation, so it is preferable to know where it is so that such adverse effects can be avoided. Esophagus monitoring is described, for example, in

International Patent Application No. PCT IB2017/057185, filed on Nov. 16, 2017, and entitled “ESOPHAGUS POSITION DETECTION BY ELECTRICAL MAPPING”, the contents of which are incorporated herein by reference in their entirety. Optionally, the esophagus position continues to be monitored during the treatment itself (the esophagus can move and/or be moved, e.g., by swallowing actions of the patient, and/or by direct surgical manipulation if required). A potential advantage of monitoring esophagus position from within the heart is to avoid use of a probe positioned within the esophagus itself.

Insofar as the procedure overall is simplified (e.g., fewer simultaneous catheters, elimination of external imaging devices, automatic visualization of targets and safety issues), a requirement for assisting staff in the procedure is potentially reduced; and potentially to the degree that a single user can perform all tasks related to data acquisition and procedure progress monitoring.

In another example, if crossing from the left atrium into the right atrium, the details mapped may comprise, for example, any of the right atrium features already discussed in relation to the right atrium acting as the first body cavity. In another example—navigation and mapping of the urinary tract—the status of the renal pelvis may be the target of mapping and assessment (for example, inspection of a kidney stone to be removed by a suitable method of lithotripsy such as laser lithotripsy).

It is noted again that each of the above-described mapping and/or assessment methods may be carried out using electrical field sensing from an electrode probe 11.

At block 1221, in some embodiments, a therapeutic delivery plan is determined, in view of the chamber geometry and tissue state as mapped and assessed in block 1220. Elements which are optionally part of the plan are detailed in relation to block 1222, which carries out the plan. Design of a plan for ablation to treat atrial fibrillation is also described, for example, in U.S. patent application Ser. No. 15/570,341 filed Oct. 29, 2017 and entitled “CALCULATION OF AN ABLATION PLAN”, the contents of which are described herein in their entirety.

At block 1222, in some embodiments, a therapy is delivered. Once again, monitoring of any of the tasks described is optionally carried out using one or more electrical field-based sensing methods. The treatment itself may also be an electrical treatment, for example, RF ablation therapy to lesion tissue.

Determination of the position of the aorta and the esophagus was described in relation to block 1220, and since the esophagus, in particular, may move during a procedure, such monitoring is optionally continued during therapy itself. Another effect which may be monitored beyond the heart wall in a similar fashion is pericardial effusion (which might be accidentally caused during a procedure, e.g., due to bleeding). Effects on local electrical field gradient due to pericardial effusion may be caused, for example, by the buildup of a thicker layer of heart-external fluid, and potentially by unusual variations in this thickness as heartbeat motions under fluid pressure constraint cause the heart to move abnormally within the pericardial membrane. It is a potential advantage to use electrical field-based pericardial effusion detection to avoid, e.g., external ultrasound imaging which might be otherwise used as an alternative.

Also optionally during an ablation procedure, cardiac wall thickness is monitored, as described in relation to block 1220. Ablation to electrically isolate tissue should create lesions which are transmural, so knowing the thickness provides a potential advantage for guiding and/or verifying the choice of ablation parameters. Where pulmonary vein blood flow may be inadvertently affected during a procedure, electrical monitoring of blood flow in the vicinity of a pulmonary vein may also be performed.

Electrical sensing data may also be used, in some embodiments, as inputs to one or more estimators used to estimates the likely effectiveness (e.g., outcome) of a procedure; either the procedure as a whole (e.g., is a whole pulmonary vein isolated by ablation), or any suitable portion of a procedure, such as a single ablation location (e.g., is the single ablation transmural) or a segment of an ablation line comprising a plurality of adjacent ablation locations (e.g., are the ablation locations placed close enough to prevent transmission between them, given the parameters of the ablations individually). Such estimators are described, for example, in International Patent Application No. PCT IB2017/057186 filed Nov. 16, 2017 and entitled “ESTIMATORS FOR ABLATION EFFECTIVENESS”, the contents of which are incorporated herein by reference in their entirety.

Optionally, electrical isolation is validated by directly sensing whether or not transmission remains after ablation. However, the possibility of temporary block due, e.g., to transient edema, potentially limits the interpretation of such sensing as relating to long-term prognosis.

Insofar as any of these methods of monitoring are performed as part of the treatment activities as such (e.g., using as electrode probe 11 the same probe that is also performing ablation using ablation element 8), there is a potential advantage for speeding the overall procedure, which, in the case of ablation, can also help reduce the effects of induced edema. Induced edema is triggered, for example, by ablation, but spreads to neighboring regions over the course of several minutes. By changing tissue state, edema may interfere with the effectiveness of ablation.

### Electrical Field Imaging Use Cases—Guidewire and Imaging Electrodes

Reference is now made to FIG. 17, which schematically illustrates a guidewire 1100 equipped with electrodes 1101, according to some embodiments of the present disclosure. In some embodiments, the guidewire is used for performing of coronary studies and/or procedures, and is optionally used to provide electrical imaging at a coronary target, and/or at one or more stages along the way to reaching a target, for example as described in relation to FIGS. 15A-15C.

In some embodiments, guidewire 1100 is a device used as an initially inserted and/or advanced (e.g., intraluminally advanced) device, along which another device, such as a catheter, is guided. In some embodiments, guidewire 1100 comprises a long (e.g. 220 cm, optionally in a range from about 20-240 cm), thin (e.g., about 0.032″ diameter) member, comprising one or more distally located clusters 1104, 1106 of electrodes 1101. In some embodiments, the electrode length is about 0.5 mm.

Electrode cluster 1104 comprises a group of one or more electrodes 1101 (optionally 1, 3, 5, 10, or 20 electrodes, or another number of electrodes) positioned along a flexible, remotely steerable tip 1102 of guidewire 1100. In some embodiments, an inter-electrode spacing between tip electrodes is about 1 mm.

In some embodiments, steerable tip 1102 is configured to be bendable, for example, through a range of curvature between straight and a 3 mm radius curvature.

Electrode cluster 1106 comprises a group of electrodes 1101 (optionally four electrodes, or another number of electrodes) positioned along a shaft region of guidewire 1100 proximal to the steerable tip 1102. In some embodiments, an inter-electrode spacing between tip electrodes is about 2 mm.

In some embodiments, electrodes 1101 are formed from electrode wires, each separately coated with an insulating material, and exposed at the positions shown. Optionally, the electrode wires serve as part of a wire braiding and/or coil structure which makes up the main length of the guidewire 1100. Additionally or alternatively, the electrode wires extend longitudinally via a central lumen of guidewire 1100.

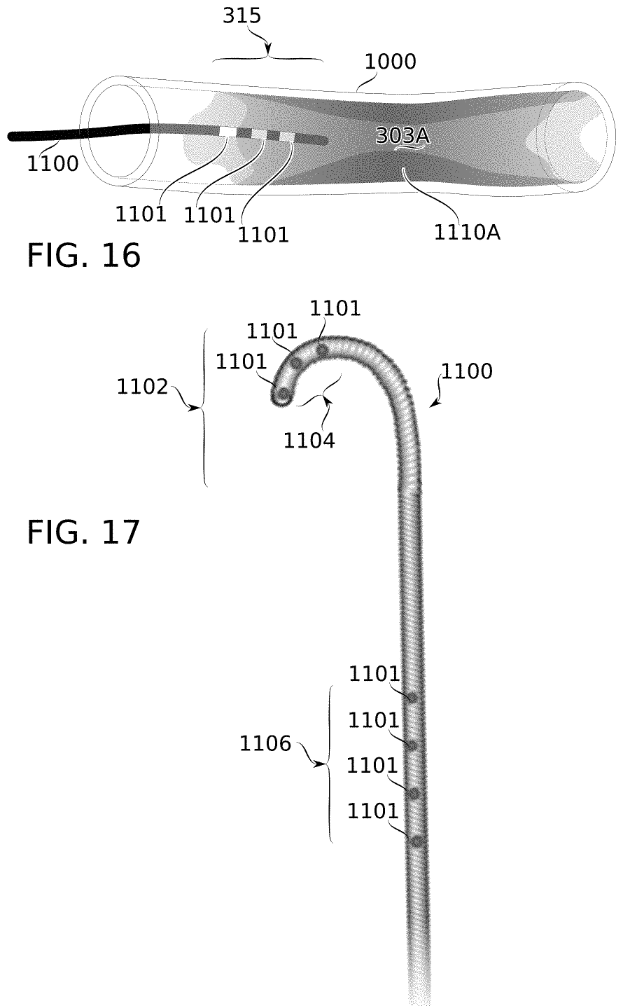

Reference is now made to FIGS. 15A-15C, which schematically illustrate stages in the insertion to a body of an electrode-equipped guide-wire 1160 configured for electrical imaging, according to some embodiments of the present disclosure. Reference is also made to FIG. 16, which schematically illustrates an electrode-equipped guidewire 1100 configured for electrical imaging, shown in relation to a stenotic blood vessel 1000. Further reference is now made to FIG. 18, which is a flowchart describing use of a guidewire 1100 for imaging, according to some embodiments of the present disclosure.

A self-imaging guide wire provides a potential advantage for use in a microcatheter procedure, by allowing the device itself to detect its surroundings, optionally in sufficient detail to support decision making at one or more key junctures of the procedure. In some embodiments, the guidewire image is potentially of sufficient quality as to obviate a need to activate another imaging modality, such as an X-ray imaging modality. In some embodiments, an entire catheter procedure is performed without any use of contrast medium injection. In some embodiments, a catheter procedure is performed without any use of contrast medium injection to achieve placement of a guidewire or catheter at a target. Optionally, contrast medium injection is used to verify results of a treatment such as stent placement and/or blood vessel opening. Optionally, treatment results are verified by use of a flow or pressure sensor.

It should be understood that in some embodiments, the imaging is performed (additionally or alternatively to using electrodes on a guidewire) using electrodes carried on another component used in catheterization, for example, a microcatheter component which is advanced over the guidewire.

At block 1202 (of FIG. 12), in some embodiments, a blood vessel insertion point for a guidewire is detected by imaging using electrodes 1101 of guidewire 1100, for example as illustrated in FIG. 15A.

In FIG. 15A, guidewire 1100 (bearing electrodes 1101) is about to be inserted into a large blood vessel 900, for example a femoral artery. In some embodiments, this represents an example of the relative positions of a sensing region 315, tissue 302A (outside of blood vessel 900) and a target feature 303A (blood vessel 900 itself). This configuration may be useful, for example, to assist in localization of an insertion point 902 for a guidewire 1100. Optionally, measurement for imaging is performed while moving the guidewire above (external to) the region of a planned insertion point, and the received measurements (from electrodes 1101) used to generate an image of the position of the target blood vessel in the region. The imaging may comprise, for example, measurement of electrical field distortions indicative of the presence of the target blood vessel, quality of the blood vessel wall (e.g., not excessive scarred by previous insertions), and/or a shape and/or orientation of the target blood vessel (e.g., a blood vessel region comprising a suitable bend for receiving guidewire 1100 at an angle that allows guidewire 1100 to be reoriented for proceeding along the blood vessel without introduction of force which is potentially injurious). Optionally, the electrical field is generated from the electrodes 1101 of guidewire 1100, or by another configuration of electrodes, for example, body surface electrodes 5.

At block 1204, in some embodiments, guidewire 1100 is inserted into selected insertion point 902. In FIG. 15B, for example, guidewire 1100 has passed into blood vessel 900, and is imaging ahead of itself (toward target feature 303A, from measurements made in region 315) as it is advanced along blood vessel 900. In this case, target feature 303A is a normal-appearing extent of blood vessel. Again, the imaging is performed using measurements by guidewire electrodes 1101.

At block 1206, in some embodiments, guidewire 1100 is advanced through the vascular system, e.g., from large blood vessel 900 to a blood vessel 900A (FIG. 15C). In FIG. 15C, guidewire 1100 is imaging ahead of itself (via measurements made using electrodes 1101) to a region 303A which comprises a partial stenotic block at a vascular junction, from measurements made in region 315) as it is advanced along blood vessel 900.

At block 1208, in some embodiments, an impediment or complication to guidewire passage is identified using guidewire imaging. For example, imaged target feature 303A potentially comprises or both of the branches ahead (branch vessels 900B and 900C), and/or parts of stenotic region 901, for example, a region comprising arterial plaque. The branch may itself be an imaged complication (with or without stenosis), based on the geometry of the region, which is to be traversed along a specific branch by the guidewire 1100. In some embodiments, an impediment of a body lumen may be encountered in the form of a growth (e.g., a tumorous growth). In some embodiments, guidewire passage may be along a vein, which may include complications in the form of tortuous branching patterns, and/or impediments in the form of partial collapse. In some embodiments, the impediment (plaque or growth, for example) is a target of the catheter procedure, in the sense that a route is being examined (tested, e.g., by electrical field sensing, imaging, and/or reconstruction) for the existence of such impediments.

In another example, illustrated in FIG. 16, guidewire 1100, advancing along a blood vessel 1000, is used while electrodes 1101 are moving to perform imaging measurements of electrical fields affected by passage through stenotic imaged region 303A.

In some embodiments, an obstruction (e.g., as in FIG. 15C or FIG. 16) is encountered within about 20 cm of an insertion position of guidewire 1100. In some embodiments, the obstruction is encountered within about 5 cm, 10 cm, 15 cm, or 25 cm.

At block 1209, in some embodiments, the impediment identified at block 1208 is passed, guided by guidewire imaging. For example, in some embodiments, the image produced is optionally used to help determine how to position catheter 1100 (e.g., by manipulation of steerable tip 1102) in order to transit and/or treat stenosis 901, 1110A.

### Electrical Field Imaging Use Cases—Needle, Guidewire and EP Catheter

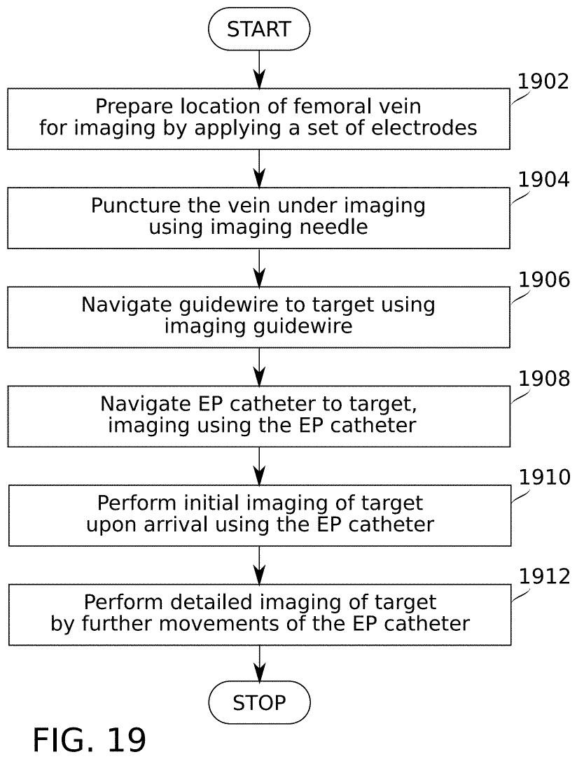

Reference is now made to FIG. 19, which is a flowchart describing use of various electrode-based imaging tools during the course of a medical procedure, according to some embodiments of the present invention. At an introducing stage of a catheter procedure, an imaging needle or other electrode-bearing device can be used for imaging to assist in introduction of a guidewire to a body, without the use of X-ray radiation and/or contrast agent injection. Examples of such electrode imaging devices are discussed, for example, in U.S. Provisional Patent Application No. 62/667,653 entitled “VERSATILE IMAGING”, filed on May 7, 2018; the contents of which are incorporated herein by reference in their entirety.

At block 1902, in some embodiments, a location of a blood vessel (e.g., as femoral vein) is prepared for imaging by applying (e.g., to the surface of the skin) a set of electrical field-transmitting electrodes configured together with an electrical field generator, so that one or more electromagnetic fields can be transmitted through a region which includes the targeted blood vessel. A targeted blood vessel, in some embodiments, comprises an anatomical feature distinct from its surroundings in terms of its effect on the bending of electrical fields in its vicinity, allowing it become a potential target for electrical field imaging. Larger diameter blood vessels, such as the femoral vein, potentially have larger effects on electrical field bending.

At block 1904, in some embodiments, an electrode-bearing imaging needle is brought into the vicinity of the femoral vein for imaging: that is, brought to a region outside the surface of the skin and within the electrical field which is transmitted from the electrical field-transmitting electrodes. The imaging needle is then moved around outside the body while electrical field measurements are recorded. Positions of the needle during measurement are optionally determined using a position monitoring system; for example, using optical tracking, priorly available knowledge (e.g., simulations) about the general structure of the transmitted electrical field, and/or constraints determined by the arrangement of electrodes on the imaging needle such as their distances from each other. The electrical field measurements are converted into a reconstruction of measurements and their positions. This in turn is converted into an image of anatomical features outside the measured region which influence distortions (bending) in the electrical field inside the measured region.

Imaging using the imaging needle continues until the location of the femoral vein is identified. Then, optionally under image guidance, the imaging needle is inserted to the femoral vein. Alternatively, the penetration is by a non-imaging needle (or by non-imaging introducer).

Optionally, imaging continues during insertion. It should be noted, however, that the needle insertion under image guidance is somewhat different from the imaging; insofar as the needle is introduced, in this stage, into the area being imaged, rather than continuing to image it remotely. For example, the needle position is tracked (e.g., by position monitoring system) compared to positions of anatomical features known from the imaging.

Optionally, tracking during insertion uses the image as a position tracking reference: since the image production includes, in some embodiments, making inferences about electrical field properties in the imaged region, information about the needle position can potentially be inferred from what it measures electrically as it enters that imaged region.

It may be noted that imaging to find the femoral vein (or another blood vessel) using an imaging needle is optionally a replacement for blood vessel-locating imaging using a guidewire.

In some embodiments, use of an imaging needle is omitted, and the procedure begins at block 1906 (e.g., after insertion of a guidewire introducer such as a needle in some other fashion).

At block 1906, in some embodiments, an imaging guidewire 1100 (that is a guidewire 1100 including electrodes 1101 configured for measuring electrical fields) is inserted to a blood vessel from the entry point discovered using the imaging needle. Optionally, the imaging guidewire 1100 is inserted to the blood vessel through the imaging needle of block 1304. Imaging guidewire 1100 is navigated to a more distal target body cavity of the procedure (e.g., navigated to enter a heart), while making measurements which are used to produce further images. The electrical fields measured are optionally transmitted from body surface electrodes, transmitted from electrodes on another probe inserted to the body, and/or transmitted from electrodes of the guidewire itself.

In some embodiments, use of an imaging guidewire is omitted, and the procedure begins at block 1308 (e.g., after guiding the guidewire in some other fashion to a target).

At block 1908, in some embodiments, an electrophysiology catheter (EP catheter) is navigated to the target body cavity reached by the guidewire. In some embodiments, progress of the EP catheter is monitored by making electrical field measurements from electrodes of the EP catheter, and optionally doing one or more of the following:
Locating the EP catheter by matching EP catheter measurements to measurements obtained during the navigation of the imaging guidewire 1100.
Locating the EP catheter by matching new images made from EP catheter measurements to images obtained during the navigation of the imaging guidewire 1100.
Locating the EP catheter by matching EP catheter measurements to measurements expected in regions imaged during the navigation of the imaging guidewire 1100.
Locating the EP catheter by integrating EP catheter measurements into one or more images obtained during navigation of the imaging guidewire 1000.

Optionally, the measurements by the EP catheter electrodes are used to enhance the images already made during navigation of the imaging guidewire

At block 1910, in some embodiments, the EP catheter reaches the procedure's target body cavity, which may comprise, for example, one or more heart chambers, such as a right atrium and/or left atrium. The EP catheter is now used for imaging, for example, using a mapping procedure such as described in International Patent Application No. PCT IB2018/056158 filed Aug. 16, 2018; and entitled FIELD GRADIENT-BASED REMOTE IMAGING, the contents of which are included herein by reference in their entirety. Initial imaging, in some embodiments, comprises making measurements by movements of a probe end of the EP catheter bearing the electrodes through a target body cavity to traverse regions near a central region of the target body cavity.

At block 1912, in some embodiments, the EP catheter continues imaging based on movements of the EP catheter which visit regions of the target body cavity in more detail.

### General

It is expected that during the life of a patent maturing from this application many relevant position tracking methods will be developed; the scope of the term “position tracking” is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1. A method of guiding a catheterization procedure in a patient using electrical field sensing by an electrode probe, the method comprising:
measuring effects of electrical field interaction with tissue of the patient using the electrode probe;
generating one or more images of the surroundings of the probe using said measured effects;
generating guidance, including said one or more images of the surrounding of the probe, based on the measuring and indicative of information providing a reference for user actions moving the electrode probe from an insertion location into the patient to a target along a planned catheter route; and
displaying the guidance, including the one or more images, during the measuring.

2. The method of claim 1, wherein the target is in a body cavity, and the guidance indicates a shape of the body cavity determined using the measuring.

3. The method of claim 1, wherein the measuring is performed as the electrode probe moves from the insertion location into the patient to the target.

4. The method of claim 3, wherein the measuring comprises measuring of a vascular obstruction, and the displayed guidance indicates the vascular obstruction.

5-6. (canceled)

7. The method of claim 1, wherein the catheterization is for treating the target.

8. (canceled)

9. The method of claim 1, wherein the target is in a body cavity and the displaying comprises displaying guidance for application of treatment in the body cavity.

10. (canceled)

11. The method of claim 1, wherein the guidance is adapted according to a current position of the probe, using the measuring.

12. The method of claim 2, wherein the shape is determined without the use of an imaging sensor external to the patient.

13-16. (canceled)

17. The method of claim 1, comprising analyzing gradients of the electrical fields measured by the electrode probe to generate the one or more images.

18. The method of claim 1, carried out without using X-ray radiation during the measuring.

19. The method of claim 2, comprising reconstruction of a 3-D shape of the body cavity to determine the shape of the body cavity.

20. The method of claim 1, comprising reconstructing body tissue lumen shapes from position data, generated using the electrode probe, during the displaying guidance for movement, wherein all position data used in the reconstructing are obtained from the measuring.

21. The method of claim 1, wherein the electrode probe is at a distal end of a catheter or guidewire.

22. The method of claim 1, wherein at least some of the electrical fields measured in the measuring are transmitted by electrodes of the electrode probe.

23. The method of claim 20, wherein the position data indicate positions of the electrode probe.

24. The method of claim 1, wherein the displaying guidance for movement is performed without the use of contrast medium.

25. (canceled)

26. The method of claim 4, wherein the vascular obstruction comprises at least one of the group consisting of:
plaque,
a vascular stenosis, and
a growth.

27. The method of claim 1, wherein the measuring is performed as the electrode probe moves from the insertion location into the patient to the target, and comprises measuring of a vascular branch, and the displayed guidance indicates the vascular branch.

28. The method of claim 1, wherein the target is in a chamber of a heart.

29. The method of claim 1, wherein the measuring comprises measuring during each of:
introduction of the probe to tubular lumen;
navigation of the probe to a vascular branch or vascular obstruction; and
navigation of the probe past the vascular branch or vascular obstruction to a body cavity comprising the target.

30. The method of claim 29, comprising withdrawing the probe from the patient, wherein no X-ray radiation is used from a time of insertion of the probe to the patient to the withdrawing.

31-61. (canceled)
